(19) United States
(12) Patent Application Publication
(10) Pub. No.: US 2026/0079144 A1
(43) Pub. Date: Mar. 19, 2026

(54) EDIBLE OIL DEGRADATION INFORMATION PROCESSING DEVICE, EDIBLE OIL DEGRADATION INFORMATION PROCESSING SYSTEM, AND EDIBLE OIL DEGRADATION INFORMATION PROCESSING METHOD

(71) Applicant: J-OIL MILLS, Inc., Tokyo (JP)

(72) Inventors: Kenichi KAKIMOTO, Tokyo (JP); Ryohei WATANABE, Tokyo (JP); Eri NUTAHARA, Tokyo (JP); Moeka ONO, Tokyo (JP)

(21) Appl. No.: 19/109,901

(22) PCT Filed: Oct. 13, 2023
(86) PCT No.: PCT/JP2023/037238

§ 371 Date: Mar. 7, 2025

(30) Foreign Application Priority Data
Nov. 8, 2022 (JP) 2022-178916

(51) Int. Cl. G01N 33/03; A47J 37/12

(52) U.S. Cl. CPC G01N 33/03; A47J 37/1266

(57) ABSTRACT

A degradation information processing device for frying oil P comprises: a storage section 73 configured to retain a plurality of pieces of degradation indicator related information about the frying oil P, which is associated with store information about a store using the frying oil P; an information acquisition section 71 configured to acquire the store information; an information selection section 74A, 74B configured to select the degradation indicator related information corresponding to the store information, from among the plurality of pieces of degradation indicator related information stored in the storage section 73; a degradation information processing section 75A, 75B configured to process degradation information about the frying oil P, based on the degradation indicator related information selected by the information selection section 74A, 74B; and a result output section 76 configured to output a processing result processed by the degradation information processing section 75A, 75B.

### TECHNICAL FIELD

The present invention relates to an edible oil degradation information processing device, an edible oil degradation information processing system, and an edible oil degradation information processing method.

### BACKGROUND ART

Deep-fry cooking, in which ingredients are deep fried using edible oil, is widely known as one of a number of cooking methods. For providing fried foods obtained by deep-fry cooking with stable qualities, it is necessary to appropriately control the quality of edible oil (hereinafter, referred to as “frying oil”) used in the deep-fry cooking. Accordingly, in particular, in some restaurants and stores which provide customers with fried foods, for the purpose of preventing the timing of wasting the frying oil from being missed, the degree of degradation of frying oil (hereinafter, simply referred to as “degradation degree”) is measured to compare the measured degree of degradation of the frying oil with a preset criterion value for disposal of oil, so as to determine or predict the degradation of the frying oil.

For example, Patent Literature 1 discloses a fat and oil degradation degree measurement device for measuring the degree of degradation of target fat and oil, which is configured to simultaneously capture an image of a color test piece immersed in the target fat and oil and an image of a color bar including a plurality of colors corresponding to the levels of acid value using a camera, calculate RGB color information on the color test piece and RGB color information on the color bar based on the captured images, measure the acid value of the target fat and oil based on the color test piece RGB color information calculated by referring to the acid value corresponding to the calculated color bar RGB color information, and determine whether the measured acid value of the target fat and oil is equal to or more than a preset acid value threshold.

Furthermore, the fat and oil degradation degree measurement device according to Patent Literature 1 is configured to retain a plurality of pieces of past determination result data, so that the time at which the acid value of the target fat and oil reaches or exceeds the acid value threshold, that is, the timing for wasting the target fat and oil can be predicted based on history information about the target fat and oil obtained by referring to the plurality of pieces of determination result data.

### CITATION LIST

### Patent Literature

Patent Literature 1: JP-A-2020-38207

### SUMMARY OF INVENTION

### Technical Problem

A criterion for determining the speed of degradation or the degradation of edible oil differs depending on the environment and state in which the edible oil is being used. However, in the method according to Patent Literature 1, the timing for wasting the target fat and oil is predicted only based on the plurality of pieces of past determination result data, in which the usage environment and state of the target fat and oil for each of the pieces of past determination result data are not considered. This makes it difficult to accurately predict the timing for wasting the target fat and oil, and thus leads to a possibility that even fat and oil that can still be used are wasted, or fat and oil that have already passed the time for wasting are still be used.

Therefore, an object of the present invention is to provide an edible oil degradation information processing device, an edible oil degradation information processing system, and an edible oil degradation information processing method, which are capable of predicting and determining the degradation of edible oil with high accuracy.

### Solution to Problem

[1] An edible oil degradation information processing device according to the present invention comprises: a storage section configured to retain a plurality of pieces of degradation indicator related information which is information about a degradation indicator of an edible oil, the degradation indicator related information being associated with store information which is information about a store using the edible oil; an information acquisition section configured to acquire the store information; an information selection section configured to select the degradation indicator related information corresponding to the store information acquired by the information acquisition section, from among the plurality of pieces of degradation indicator related information stored in the storage section; a degradation information processing section configured to process degradation information which is information about degradation of the edible oil, based on the degradation indicator related information selected by the information selection section;

and a result output section configured to output a processing result of the degradation information processed by the degradation information processing section.

[2] Preferably, in the edible oil degradation information processing device according to [1], the store information includes: business type information indicative of a type of business of the store; cooking tool information which is information about a cooking tool used for cooking with the edible oil; oil type information indicative of a type of the edible oil; and degradation speed information which is information about a degradation speed of the edible oil.

[3] Preferably, in the edible oil degradation information processing device according to [2], the degradation speed information includes: operating hour information indicative of operating hours of the cooking tool in a day; sales information indicative of sale of the store in a day; and number of tools information indicative of the number of units of the cooking tool in operation.

[4] Preferably, in the edible oil degradation information processing device according to [1], the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively, the information acquisition section is configured to further acquire data about a measured value of the degradation indicator, the information selection section is configured to: select one of the degradation characteristic models stored in the storage section, which corresponds to the store information acquired by the information acquisition section; and select one of the degradation criterion values stored in the storage section, which corresponds to the store information acquired by the information acquisition section; the degradation information processing section is configured to predict the degradation information based on the measured value of the degradation indicator acquired by the information acquisition section, and the one of the degradation characteristic models and the one of the degradation criterion values selected by the information selection section, and the result output section is configured to output a prediction result of the degradation information obtained by the degradation information processing section.

[5] Preferably, in the edible oil degradation information processing device according to [4], the degradation information includes at least one of data indicative of a remaining time until the edible oil reaches a predetermined oil disposal time point, data indicative of a timing of removing a part of the edible oil in a cooking tool used for cooking with the edible oil and adding another edible oil, which is different from the edible oil, in the edible oil, data indicative of a timing of filtering the edible oil in the cooking tool, or data indicative of an amount of remaining ingredients that can be cooked until the edible oil reaches a predetermined degradation degree.

[6] Preferably, in the edible oil degradation information processing device according to [4], the degradation characteristic model includes at least one of a model indicative of a correlation between a heating time of the edible oil and the degradation indicator, or a model indicative of a correlation between an amount of ingredients that can be cooked with the edible oil and the degradation indicator.

[7] Preferably, in the edible oil degradation information processing device according to [4], a degradation speed classification section configured to classify a degradation speed of the edible oil is further included, the information acquisition section is configured to acquire, as the store information, business type information indicative of a type of business of the store, cooking tool information which is information about a cooking tool used for cooking with the edible oil, oil type information indicative of a type of the edible oil, operating hour information indicative of operating hours of the cooking tool in a day, sales information indicative of sale of the store in a day, and number of tools information indicative of the number of units of the cooking tool in operation, the degradation speed classification section is configured to, based on the operating hour information, the sales information, and the number of tools information acquired by the information acquisition section, calculate sales at one unit of the cooking tool per hour in the store to classify the degradation speed of the edible oil into one of a plurality of stages, and the information selection section is configured to, based on the business type information, the cooking tool information, and the oil type information acquired by the information acquisition section, and the one of the plurality of stages classified by the degradation speed classification section, select one of the degradation characteristic models, which corresponds to the store information.

[8] Preferably, in the edible oil degradation information processing device according to [7], the information acquisition section is configured to further acquire, as the store information, customer name information about the store, and the degradation speed classification section is configured to classify the degradation speed of the edible oil into one of the plurality of stages, considering, relative to the sales at one unit of the cooking tool per hour in the store as calculate, at least either a capacity of the cooking tool included in the cooking tool information acquired by the information acquisition section or the customer name information.

[9] Preferably, in the edible oil degradation information processing device according to [1], the plurality of pieces of degradation indicator related information includes data about degradation criterion values, each of which is indicative of a criterion relating to the degradation indicator, respectively, the information acquisition section is configured to further acquire data about a measured value of the degradation indicator, the information selection section is configured to select one of the degradation criterion values stored in the storage section, which corresponds to the store information acquired by the information acquisition section, the degradation information processing section is configured to, based on the measured value of the degradation indicator acquired by the information acquisition section and the one of the degradation criterion values selected by the information selection section, determine whether the edible oil has reached the one of the degradation criterion values, and the result output section is configured to output, as a determination result of the degradation information, a result determined by the degradation information processing section.

Preferably, in the edible oil degradation information processing device according to [9], the degradation criterion values include at least one of an oil disposal criterion value serving as a criterion for the degradation indicator at a predetermined oil disposal time point, a removing/adding oil criteria value serving as a criterion for the degradation indicator in a case where a removing/adding oil operation of removing a part of the edible oil in the cooking tool and adding another edible oil, which is different from the edible oil, in the edible oil needs to be performed, or a filtering criterion value serving as a criterion for the degradation indicator at a time of filtering the edible oil in the cooking tool.

Preferably, in the edible oil degradation information processing device according to [1], the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil, the information acquisition section is configured to further acquire data about a measured value of the degradation indicator and remaining oil amount data which is data about a remaining oil amount of the edible oil in the cooking tool immediately before performing the removing/adding oil operation, the information selection section is configured to select one of the after-removing/adding oil criterion values stored in the storage section, which corresponds to the store information acquired by the information acquisition section, the degradation information processing section is configured to calculate a removing/adding oil amount necessary in the removing/adding oil operation, based on the measured value of the degradation indicator and the remaining oil amount data acquired by the information acquisition section, and the after-removing/adding oil criterion value selected by the information selection section, and the result output section is configured to output the removing/adding oil amount calculated by the degradation information processing section as a calculation result of the degradation information.

Furthermore, an edible oil degradation information processing system according to the present invention, comprises: an input terminal into which store information, which is information about a store using an edible oil, is input; a degradation information processing device configured to process degradation information which is information about degradation of the edible oil; and a notification device configured to notify information output from the degradation information processing device, the degradation information processing device being configured to: retain a plurality of pieces of degradation indicator related information which is information about a degradation indicator of the edible oil and associated with the store information; acquire the store information output from the input terminal; select the degradation indicator related information corresponding to the store information as acquired, from among the plurality of pieces of degradation indicator related information as stored; process the degradation information based on the degradation indicator related information as selected; and output a processing result of the degradation information as processed to the notification device.

Preferably, in the edible oil degradation information processing system according to [12], the store information includes: business type information indicative of a type of business of the store; cooking tool information which is information about a cooking tool used for cooking with the edible oil; oil type information indicative of a type of the edible oil; and degradation speed information which is information about a degradation speed of the edible oil.

Preferably, in the edible oil degradation information processing system according to [13], the degradation speed information includes: operating hour information indicative of operating hours of the cooking tool in a day; sales information indicative of sale of the store in a day; and number of tools information indicative of the number of units of the cooking tool in operation.

Preferably, in the edible oil degradation information processing system according to [12], a measurement device for measuring the degradation indicator of the edible oil is further included, the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively, and the degradation information processing device is configured to: further acquire data about a measured value of the degradation indicator measured by the measurement device; select one of the degradation characteristic models as stored, which corresponds to the store information as acquired, and also select one of the degradation criterion values as stored, which corresponds to the store information as acquired; predict the degradation information based on the measured value of the degradation indicator as acquired, and the one of the degradation characteristic models and the one of the degradation criterion values as selected; and output a prediction result of the degradation information as predicted to the notification device.

Preferably, in the edible oil degradation information processing system according to [15], the degradation information includes at least one of data indicative of a remaining time until the edible oil reaches a predetermined oil disposal time point, data indicative of a timing of removing a part of the edible oil in a cooking tool used for cooking with the edible oil and adding another edible oil, which is different from the edible oil, in the edible oil, data indicative of a timing of filtering the edible oil in the cooking tool, or data indicative of an amount of remaining ingredients that can be cooked until the edible oil reaches a predetermined degradation degree.

Preferably, in the edible oil degradation information processing system according to [15], the degradation characteristic model includes at least one of a model indicative of a correlation between a heating time of the edible oil and the degradation indicator, or a model indicative of a correlation between an amount of ingredients that can be cooked with the edible oil and the degradation indicator.

Preferably, in the edible oil degradation information processing system according to [15], the information acquisition section is configured to acquire, as the store information, business type information indicative of a type of business of the store, cooking tool information which is information about a cooking tool used for cooking with the edible oil, oil type information indicative of a type of the edible oil, operating hour information indicative of operating hours of the cooking tool in a day, sales information indicative of sale of the store in a day, and number of tools information indicative of the number of units of the cooking tool in operation, based on the operating hour information, the sales information, and the number of tools information acquired by the information acquisition section, calculate sales at one unit of the cooking tool per hour in the store to classify the degradation speed of the edible oil into one of a plurality of stages, and based on the business type information, the cooking tool information, and the oil type information acquired by the information acquisition section, and the one of the plurality of stages classified by the degradation speed classification section, select one of the degradation characteristic models, which corresponds to the store information.

Preferably, in the edible oil degradation information processing system according to [12], a measurement device for measuring the degradation indicator of the edible oil is further included, the plurality of pieces of degradation indicator related information includes data about degradation criterion values, each of which is indicative of a criterion relating to the degradation indicator, respectively, and the degradation information processing device is configured to: further acquire data about a measured value of the degradation indicator measured by the measurement device, select one of the degradation criterion values as stored, which corresponds to the store information as acquired; based on the measured value of the degradation indicator as acquired and the one of the degradation criterion values as selected, determine whether the edible oil has reached the one of the degradation criterion values, and output, as a determination result of the degradation information, a result as determined to the notification device.

Preferably, in the edible oil degradation information processing system according to [19], the degradation criterion values include at least one of an oil disposal criterion value serving as a criterion for the degradation indicator at a predetermined oil disposal time point, a removing/adding oil criteria value serving as a criterion for the degradation indicator in a case where a removing/adding oil operation of removing a part of the edible oil in the cooking tool and adding another edible oil, which is different from the edible oil, in the edible oil needs to be performed, or a filtering criterion value serving as a criterion for the degradation indicator at a time of filtering the edible oil in the cooking tool.

Preferably, in the edible oil degradation information processing system according to [12], the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil, and the degradation information processing device is configured to: acquire data about a measured value of the degradation indicator and remaining oil amount data which is data about a remaining oil amount of the edible oil in the cooking tool immediately before performing the removing/adding oil operation; select one of the after-removing/adding oil criterion values as stored, which corresponds to the store information as acquired; calculate a removing/adding oil amount necessary in the removing/adding oil operation, based on the measured value of the degradation indicator and the remaining oil amount data as acquired, and the after-removing/adding oil criterion value as selected; and output, as a calculation result of the degradation information, the removing/adding oil amount as calculated to the notification device.

Still further, according to the present invention, an edible oil degradation information processing method for processing degradation information, which is information about degradation of an edible oil, uses an input terminal into which store information, which is information about a store using the edible oil, is input, a degradation information processing device configured to process the degradation information, and a notification device configured to notify information output from the degradation information processing device, and the method comprises: information acquiring step of, by the degradation information processing device, acquiring the store information output from the input terminal; information selecting step of, by the degradation information processing device, selecting the degradation indicator related information corresponding to the store information acquired in the information acquiring step, from among the plurality of pieces of degradation indicator related information as stored; an information processing step of, by the degradation information processing device, processing the degradation information based on the degradation indicator related information selected in the information selecting step; and a processing result outputting step of, by the degradation information processing device, outputting a processing result of the degradation information processed in the information processing step to the notification device.

Preferably, in the edible oil degradation information processing method according to [22], the store information includes: business type information indicative of a type of business of the store; cooking tool information which is information about a cooking tool used for cooking with the edible oil; oil type information indicative of a type of the edible oil; and degradation speed information which is information about a degradation speed of the edible oil.

Preferably, in the edible oil degradation information processing method according to [23], the degradation speed information includes: operating hour information indicative of operating hours of the cooking tool in a day; sales information indicative of sale of the store in a day; and number of tools information indicative of the number of units of the cooking tool in operation.

Preferably, in the edible oil degradation information processing method according to [22], a measurement device for measuring the degradation indicator of the edible oil is further used, the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively, in the information acquiring step, the degradation information processing device further acquires data about a measured value of the degradation indicator measured by the measurement device, in the information selecting step, the degradation information processing device selects one of the degradation characteristic models as stored, which corresponds to the store information acquired in the information acquiring step, and also selects one of the degradation criterion values as stored, which corresponds to the store information acquired in the information acquiring step, in the information processing step, the degradation information processing device predicts the degradation information based on the measured value of the degradation indicator acquired in the information acquiring step, and the one of the degradation characteristic models and the one of the degradation criterion values selected in the information selecting step, and in the processing result outputting step, the degradation information processing device outputs a prediction result of the degradation information predicted in the information processing step to the notification device.

Preferably, in the edible oil degradation information processing method according to [25], the degradation information includes at least one of data indicative of a remaining time until the edible oil reaches a predetermined oil disposal time point, data indicative of a timing of removing a part of the edible oil in a cooking tool used for cooking with the edible oil and adding another edible oil, which is different from the edible oil, in the edible oil, data indicative of a timing of filtering the edible oil in the cooking tool, or data indicative of an amount of remaining ingredients that can be cooked until the edible oil reaches a predetermined degradation degree.

Preferably, in the edible oil degradation information processing method according to [25], the degradation characteristic model includes at least one of a model indicative of a correlation between a heating time of the edible oil and the degradation indicator, or a model indicative of a correlation between an amount of ingredients that can be cooked with the edible oil and the degradation indicator.

Preferably, in the edible oil degradation information processing method according to [25], the degradation information processing device further includes a degradation speed classifying step of classifying a degradation speed of the edible oil, in the information acquiring step, the degradation information processing device acquires, as the store information, business type information indicative of a type of business of the store, cooking tool information which is information about a cooking tool used for cooking with the edible oil, oil type information indicative of a type of the edible oil, operating hour information indicative of operating hours of the cooking tool in a day, sales information indicative of sale of the store in a day, and number of tools information indicative of the number of units of the cooking tool in operation, in the degradation speed classifying step, based on the operating hour information, the sales information, and the number of tools information acquired in the information acquiring step, the degradation information processing device calculates sales at one unit of the cooking tool per hour in the store to classify the degradation speed of the edible oil into one of a plurality of stages, and in the information selecting step, based on the business type information, the cooking tool information, and the oil type information acquired by the information acquiring step, and the one of the plurality of stages classified in the degradation speed classifying step, the degradation information processing device selects one of the degradation characteristic models, which corresponds to the store information.

Preferably, in the edible oil degradation information processing method according to [22], a measurement device for measuring the degradation indicator of the edible oil is further used, the plurality of pieces of degradation indicator related information includes data about degradation criterion values, each of which is indicative of a criterion relating to the degradation indicator, respectively, in the information acquiring step, the degradation information processing device further acquires data about a measured value of the degradation indicator measured by the measurement device, in the information selecting step, the degradation information processing device selects one of the degradation criterion values as stored, which corresponds to the store information acquired in the information acquiring step, in the information processing step, based on the measured value of the degradation indicator acquired in the information acquiring step and the one of the degradation criterion values selected in the information selecting step, the degradation information processing device determines whether the edible oil has reached the one of the degradation criterion values, and in the processing result outputting step, the degradation information processing device outputs, as a determination result of the degradation information, a result as determined in the information processing step to the notification device.

Preferably, in the edible oil degradation information processing method according to [29], the degradation criterion values include at least one of an oil disposal criterion value serving as a criterion for the degradation indicator at a predetermined oil disposal time point, a removing/adding oil criteria value serving as a criterion for the degradation indicator in a case where a removing/adding oil operation of removing a part of the edible oil in the cooking tool and adding another edible oil, which is different from the edible oil, in the edible oil needs to be performed, or a filtering criterion value serving as a criterion for the degradation indicator at a time of filtering the edible oil in the cooking tool.

Preferably, in the edible oil degradation information processing method according to [22], the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil, in the information acquiring step, the degradation information processing device further acquires data about a measured value of the degradation indicator and remaining oil amount data which is data about a remaining oil amount of the edible oil in the cooking tool immediately before performing the removing/adding oil operation, in the information selecting step, the degradation information processing device selects one of the after-removing/adding oil criterion values as stored, which corresponds to the store information as acquired, in the information processing step, the degradation information processing device calculates a removing/adding oil amount necessary in the removing/adding oil operation, based on the measured value of the degradation indicator and the remaining oil amount data acquired in the information acquiring step, and the after-removing/adding oil criterion value selected in the information selecting step, and in the processing result outputting step, the degradation information processing device outputs, as a calculation result of the degradation information, the removing/adding oil amount calculated in the information processing step to the notification device.

### Advantageous Effects of Invention

According to the present invention, it is possible to predict and determine the degradation of edible oil with high accuracy. The problems, configurations, and advantageous effects other than those described above will be clarified by explanation of the embodiments below.

### BRIEF DESCRIPTION OF DRAWINGS

FIG. 1 illustrates a part of a cooking area in which deep-frying is performed.

FIG. 2A illustrates a graph showing the correlation between the heating time and color of frying oil, for different sales at a store.

FIG. 2B illustrates a graph showing the correlation between the heating time and acid value of frying oil, for different sales at a store.

FIG. 3A illustrates a graph showing the correlation between the heating time and color of frying oil, for different types of fried chickens.

FIG. 3B illustrates a graph showing the correlation between the heating time and acid value of frying oil, for different types of fried chickens.

FIG. 4A illustrates a graph showing the correlation between the heating time and color of frying oil, for different capacities of an oil vat.

FIG. 4B illustrates a graph showing the correlation between the heating time and acid value of frying oil, for different capacities of an oil vat.

FIG. 5A illustrates a graph showing the correlation between the heating time and color of frying oil, for different oil types.

FIG. 5B illustrates a graph showing the correlation between the heating time and acid value of frying oil, for different oil types.

FIG. 6 illustrates a graph showing the correlation between the number of fried foods and the acid value of frying oil, for different types of deep-frying materials.

FIG. 7 is a system configuration diagram illustrating a configuration example of a degradation information processing system for frying oil.

FIG. 8 illustrates an example of a hardware configuration of a degradation information processing server.

FIG. 9 is a functional block diagram illustrating functions of a degradation information processing server.

FIG. 10A illustrates a degradation indicator related map stored in a storage section, which is an example of using color of frying oil as a degradation indicator.

FIG. 10B illustrates a degradation indicator related map stored in a storage section, which is an example of using an acid value of frying oil as a degradation indicator.

FIG. 10C illustrates a map of an example of classification of degradation speed.

FIG. 11 illustrates a flowchart of an overall flow of the processing to be executed by a degradation information processing server.

FIG. 12 illustrates a flowchart of a flow of degradation prediction processing.

FIG. 13 illustrates a flowchart of an example of a flow of degradation determination processing.

### DESCRIPTION OF EMBODIMENTS

Hereinafter, as an aspect of an edible oil degradation information processing system according to an embodiment of the present invention, a system applicable to cooking of fried foods such as fried chickens, croquettes, and karaage, which is performed, for example, in convenience stores and supermarkets.

Cooking of fried foods is referred herein as “deep-frying”, edible oil to be used in deep-frying is referred herein as “frying oil”, and ingredients to be deep fried is referred herein as “deep-frying material”.

### (Arrangement in Cooking Area 1)

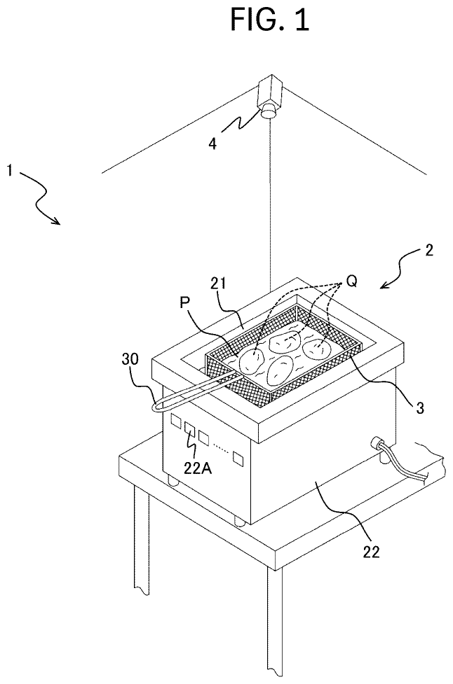

Firstly, an example of an environment in which deep-frying is performed will be described with reference to FIG. 1.

FIG. 1 illustrates a part of a cooking area 1 in which deep-frying is performed.

For example, in a store such as a convenience store or a supermarket, the cooking area 1 in which deep-frying is performed is provided in the store so as to provide customers with freshly deep-fried foods. Within the cooking area 1, as a cooking tool to be used in deep-frying, for example, an electric fryer 2 is installed.

The fryer 2 includes an oil vat 21 for holding frying oil P therein, and a housing 22 for accommodating the oil vat 21. On a side surface of the housing 22, a plurality of switches 22A for setting the temperature of the frying oil P and the details of the deep-frying for each type of deep-frying materials Q is provided.

For performing deep-frying, firstly, a cook places the deep-frying material Q in a fry basket 3 having a handle 30, and then hooks the handle 30 on an upper end portion of the housing 22 so as to immerse, in the frying oil P, the deep-frying material Q placed in the fry basket 3. At the same time, therebefore, or thereafter, the cook operates one of the switches 22A which corresponds to the type of the deep-frying material Q which is to be cooked.

Subsequently, the fryer 2 identifies the one of the switches 22A which was operated by the cook, and when a deep-frying time, which is associated with the operated one of the switches 22A, passes, the fryer 2 notifies the cook of the completion of deep-frying. At the same time, the fry basket 3 holding the deep-fried food (deep-frying material Q after being deep fried) automatically rises from the oil vat 21 so that the deep-fried food that has been immersed in the frying oil P is pulled up.

As a technique of informing the completion of deep-frying of a fried food, for example, a buzzer sound may be output from a speaker of the fryer 2 or completion of deep-frying may be shown on a monitor installed near the fryer 2.

The cook who has noticed the completion of deep-frying pulls up the fry basket 3 to take the fried food out therefrom. The operation of pulling up the fry basket 3 from the oil vat 21 may be automatically performed by a drive mechanism which can be provided in the fryer 2.

In the present embodiment, the cooking area 1 includes a camera 4 for acquiring an image of the surface of the frying oil P in the oil vat 21, which is mounted to the ceiling which is positioned above the oil vat 21. However, the camera 4 does not necessarily have to be mounted to the ceiling positioned above the oil vat 21. The camera 4 may be mounted to any position, for example, a wall near the fryer 2, as long as it is held at a position allowing the image of the surface of the frying oil P in the oil vat 21 to be captured.

The camera 4 is a video camera for capturing a video or a still camera for capturing a still image, and is used in measurement of the color of the frying oil P.

Specifically, the color of the frying oil P is measured based on a brightness value (for example, RGB value) of an image of the surface of the frying oil P in the oil vat 21 captured by the camera 4. Thus, the image captured by the camera 4 needs to include at least the image of the surface of the frying oil P in the oil vat 21, however, it may include an image other than the image of the surface of the frying oil P, such as an image of a portion of the oil vat 21 or an image of an object (specifically, the deep-frying material Q or a portion of the fry basket 3) immersed in the frying oil P.

The color of the frying oil P gets darker as the heating time progresses, and accordingly, it is used as a degradation indicator which is an indicator indicative of the degree of degradation of the frying oil P (hereinafter, simply referred to as “degradation degree”). The degradation indicators of the frying oil P other than the color of the frying oil P are, for example, the acid value (AV) of the frying oil P, the amount of polar compounds (PC) of the frying oil P, the viscosity of the frying oil P, the rate of increase in viscosity of the frying oil P, the anisidine value of the frying oil P, the carbonyl value of the frying oil P, the smoke point of the frying oil P, the tocopherol contents of the frying oil P, the iodine value of the frying oil P, the refractive indicator of the frying oil P, the amount of volatile components of the frying oil P, the volatile component composition of the frying oil P, the flavor of the frying oil P, the amount of volatile components of a fried food obtained by deep-frying using the frying oil P, the volatile component composition of a fried food obtained by deep-frying using the frying oil P, and the flavor of a fried food obtained by deep-frying using the frying oil P.

A user who uses the frying oil P (for example, a cook, a store staff, or the like) measures the degradation indicator of the frying oil P, and based on a measured value of the degradation indicator of the frying oil P, determines or predicts the degradation of the frying oil P, so that the quality of the frying oil P and the qualities of the fried foods obtained by deep-frying using the frying oil P can be maintained.

Each of the degradation indicators of the frying oil P can be measured using a measurement device such as the camera 4 or various sensors. For example, for measurement of the acid value (AV) of the frying oil P, a test paper for measuring the acid value based on the change in color of a portion on which the frying oil P is dropped, a measurement apparatus which is to be immersed in the frying oil P so as to directly measure the acid value of the frying oil P, or the like may be used. For measurement of the amount of polar compounds (PC) of the frying oil P, a measurement apparatus which is to be immersed in the frying oil P so as to directly measure the amount of polar compounds contained in the frying oil P or the like may be used. For measurement of the amount of volatile components and volatile component composition of the frying oil P and measurement of the amount of volatile components and volatile component composition of the fried food obtained by deep-frying using the frying oil P, a commonly-used gas sensor (for example, semiconductor gas sensor or crystal oscillator gas sensor) or the like may be used.

The techniques of measuring the degradation indicators other than the ones mentioned above include, for example, a technique of recognizing an image captured by the camera 4 by means of an image recognition technique to identify the type and number of fried foods obtained by deep-frying using the frying oil P, and predicting each degradation indicator based on the correlation between the type and number of fried foods and each degradation indicator, a technique of measuring the spectrum of the frying oil P by means of a spectrometer and predicting each degradation indicator based on the correlation between the spectrum of the frying oil P and each degradation indicator, and a technique of predicting each degradation indicator based on the correlation between the number of times the setting switches 22A of the fryer 2 have been operated (that is, the number of times the fried foods have been cooked) and each degradation indicator.

A measurement device and measurement technique for measuring the degradation indicator of the frying oil P are not necessarily limited to the ones described above, but other known measurement devices and measurement techniques may be used.

### (Degradation Characteristics of Frying Oil P)

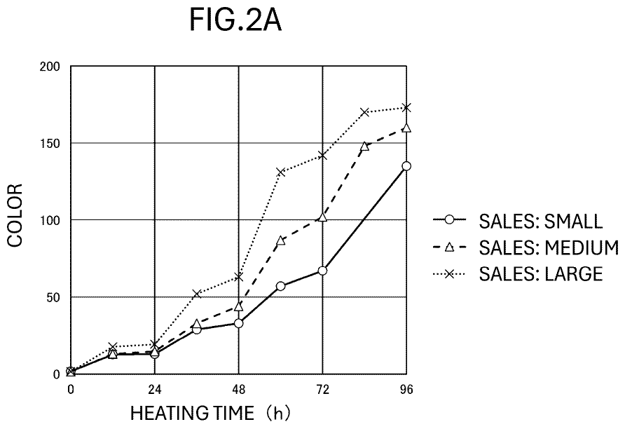

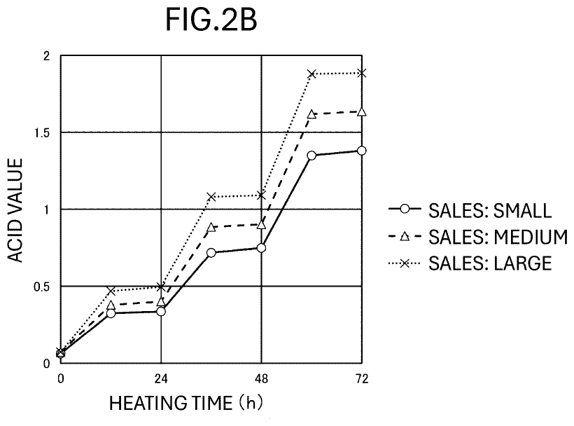

Next, the degradation characteristics of the frying oil P will be described with reference to FIG. 2 to FIG. 6.

The progression of degradation of the frying oil P, in other words, the speed of degradation of the frying oil P is not constant, but it varies depending on the environment and state in which the frying oil P is being used. In other words, the degradation characteristics of the frying oil P cannot be defined to one, but include a plurality of characteristics depending on the environment and state in which the frying oil P is to be used.

For example, the degradation speed of the frying oil P varies depending on the amount of the deep-frying material Q (the number of the deep-frying material Q).

FIG. 2A illustrates the correlation between the heating time and color of the frying oil P for different sales at a store. FIG. 2B illustrates the correlation between the heating time and acid value of the frying oil P for different sales at a store.

FIG. 2A illustrates three patterns of the correlation between the heating time and color of the frying oil P for the cases where the sales of fried foods at a store are “small”, “medium”, and “large”, respectively. In FIG. 2A, a graph shown with a solid line connecting ∘ marks represents the correlation for “small” sales, a graph shown with a broken line connecting A marks represents the correlation for “medium” sales, and a graph shown with a dotted line connecting x marks represents the correlation for “large” sales, respectively.

FIG. 2B illustrates three patterns of the correlation between the heating time and acid values of the frying oil P for the cases where the sales of fried foods at a store are “small”, “medium”, and “large”, respectively. In the same manner as FIG. 2A, in FIG. 2B, a graph shown with a solid line connecting ∘ marks represents the correlation for “small” sales, a graph shown with a broken line connecting Δ marks represents the correlation for “medium” sales, and a graph shown with a dotted line connecting x marks represents the correlation for “large” sales, respectively.

As illustrated in FIG. 2A and FIG. 2B, the frying oil P in the case of the “medium” sales gets deteriorated faster than the case of the “small” sales, and moreover, that in the case of the “large” sales gets deteriorated faster than the case of the “medium” sales.

For example, in FIG. 2A, the heating time until the color value of the frying oil P reaches 100 is about 84 hours in the case of the “small” sales, it is about 72 hours in the case of the “medium” sales, and it is about 54 hours in the case of the “large” sales. In FIG. 2B, the heating time until the acid value of the frying oil P reaches 1 is about 54 hours in the case of the “small” sales, it is about 51 hours in the case of the “medium” sales, and it is about 35 hours in the case of the “large” sales.

That is, the larger the sales of the fried foods in a store are, the faster the degradation speed of the frying oil P becomes, and thus the degradation characteristics of the frying oil P in this case shows the characteristics in which it gets deteriorated more easily. The degradation speed of the frying oil P differs depending on the sales of fried foods at a store even if the other conditions (the capacity of the oil vat 21, the type of the deep-frying material Q, the type of the frying oil P, the amount of fried foods obtained by deep-frying using the frying oil P, and the like) are the same.

The sales of fried foods at a store correspond to the amount of the deep-frying material Q (fried foods) which were deep-fried using the frying oil P, and accordingly, the amount of the deep-frying material Q is “small” in the case of the “small” sales, the amount of the deep-frying material Q is “medium” in the case of the “medium” sales, and the amount of the deep-frying material Q is “large” in the case of the “large” sales.

Thus, the degradation characteristics (degradation speed) of the frying oil P differ depending on the sales of fried foods at a store, that is, depending on the amount of the material Q which were deep-fried using the frying oil P.

Furthermore, the degradation speed of the frying oil P varies depending on, for example, the type of the deep-frying material Q.

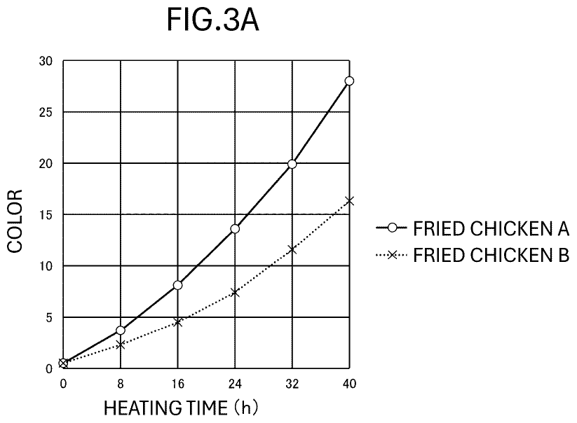

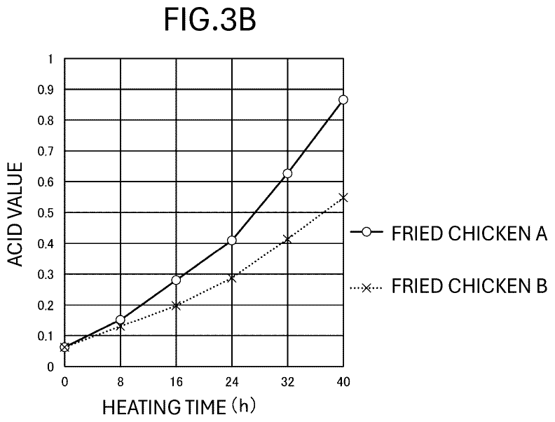

FIG. 3A illustrates a graph showing the correlation between the heating time and color of the frying oil P, for fried chickens which differ in their types. FIG. 3B illustrates a graph showing the correlation between the heating time and acid value of the frying oil P, for fried chickens which differ in their types.

FIG. 3A illustrates two patterns of the correlation between the heating time and color of the frying oil P for the cases where, as the deep-frying materials Q, two different types of fried chicken A and fried chicken B are placed in the frying oil P, respectively. In FIG. 3A, a graph shown with a solid line connecting ∘ marks represents the correlation for deep-frying the fried chicken A, and a graph shown with a dotted line connecting x marks represents the correlation for deep-frying the fried chicken B, respectively.

FIG. 3B illustrates two patterns of the correlation between the heating time and acid values of the frying oil P for the cases where, as the deep-frying materials Q, two different types of the fried chicken A and the fried chicken B are placed in the frying oil P, respectively. In the same manner as FIG. 3A, in FIG. 3B, a graph shown with a solid line connecting ∘ marks represents the correlation for deep-frying the fried chicken A, and a graph shown with a dotted line connecting x marks represents the correlation for deep-frying the fried chicken B, respectively.

As illustrated in FIG. 3A and FIG. 3B, the frying oil P in the case of deep-frying the fried chicken A gets deteriorated faster than the case of deep-frying the fried chicken B.

For example, in FIG. 3A, the heating time until the color value of the frying oil P reaches 10 is about 18 hours in the case of deep-frying the fried chicken A, and it is about 28 hours in the case of deep-frying the fried chicken B. In FIG. 3B, the heating time until the acid value of the frying oil P reaches 0.5 is about 27 hours in the case of deep-frying the fried chicken A, and it is about 37 hours in the case of deep-frying the fried chicken B.

That is, even in the case of deep-frying “fried chickens” which are in the same fried food category, the degradation speed of the frying oil P differs depending on the type of materials such as the type of chicken or the type of batter. In FIG. 3A and FIG. 3B, the frying oil P in the case of deep-frying the fried chicken A gets deteriorated faster than that in the case of deep-frying the fried chicken B, and thus the degradation characteristics of the frying oil P in the former case show the characteristics in which it gets deteriorated more easily.

The degradation speed of the frying oil P differs depending on the type of the deep-frying material Q (including not only the types of ingredients making up the deep-frying material Q, but also the types of categories of the deep-frying materials Q, such as fried chickens, croquettes, and the like), even if the other conditions (the sales of fried foods at a store, the capacity of the oil vat 21, the type of the frying oil P, the amount of fried foods obtained by deep-frying using the frying oil P, and the like) are the same. Accordingly, the degradation characteristics (degradation speed) of the frying oil P differ depending on the type of the deep-frying material Q.

Furthermore, the degradation speed of the frying oil P varies depending on, for example, the capacity of the oil vat 21 of the fryer 2.

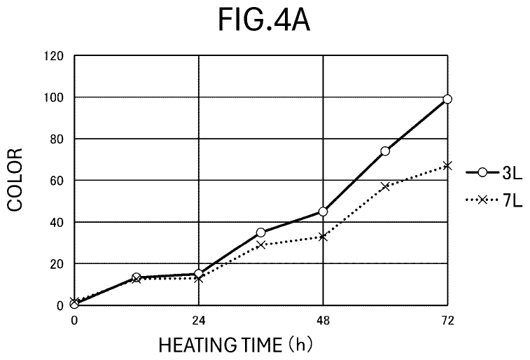

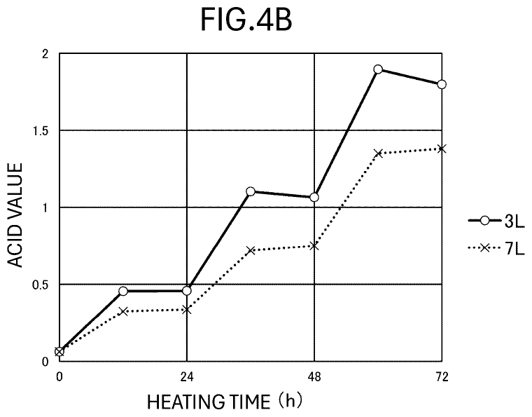

FIG. 4A illustrates a graph showing the correlation between the heating time and color of the frying oil P, for different capacities of the oil vat 21. FIG. 4B illustrates a graph showing the correlation between the heating time and acid value of the frying oil P, for different capacities of the oil vat 21.

FIG. 4A illustrates two patterns of the correlation between the heating time and color of the frying oil P for the cases where the capacities of the oil vat 21 are “3 liters” and “7 liters”, respectively. In FIG. 4A, a graph shown with a solid line connecting ∘ marks represents the correlation for the case where the capacity of the oil vat 21 is 3 liters, and a graph shown with a dotted line connecting x marks represents the correlation for the case where the capacity of the oil vat 21 is 7 liters, respectively.

FIG. 4B illustrates two patterns of the correlation between the heating time and acid value of the frying oil P for the cases where the capacities of the oil vat 21 are “3 liters” and “7 liters”, respectively. In the same manner as FIG. 4A, in FIG. 4B, a graph shown with a solid line connecting ∘ marks represents the correlation for the case where the capacity of the oil vat 21 is 3 liters, and a graph shown with a dotted line connecting x marks represents the correlation for the case where the capacity of the oil vat 21 is 7 liters, respectively.

As illustrated in FIG. 4A and FIG. 4B, the frying oil P in the case where the capacity of the oil vat 21 is 3 liters gets deteriorated faster than the case where the capacity of the oil vat 21 is 7 liters.

For example, in FIG. 4A, the heating time until the color value of the frying oil P reaches 40 is about 42 hours in the case where the capacity of the oil vat 21 is 3 liters, and it is about 51 hours in the case where the capacity of the oil vat 21 is 7 liters. In FIG. 4B, the heating time until the acid value of the frying oil P reaches is about 33 hours in the case where the capacity of the oil vat 21 is 3 liters, and it is about 54 hours in the case where the capacity of the oil vat 21 is 7 liters.

That is, the smaller the capacity of the oil vat 21 is, the faster the degradation speed of the frying oil P becomes, and thus the degradation characteristics of the frying oil P in this case shows the characteristics in which it gets deteriorated more easily. The degradation speed of the frying oil P differs depending on the capacity of the oil vat 21 even if the other conditions (the sales of fried foods at a store, the type of the deep-frying material Q, the type of the frying oil P, the amount of fried foods obtained by deep-frying using the frying oil P, and the like) are the same. Accordingly, the degradation characteristics (degradation speed) of the frying oil P differ depending on the capacity of the oil vat 21 of the fryer 2.

Furthermore, the degradation speed of the frying oil P varies depending on, for example, the type of the frying oil P (oil type).

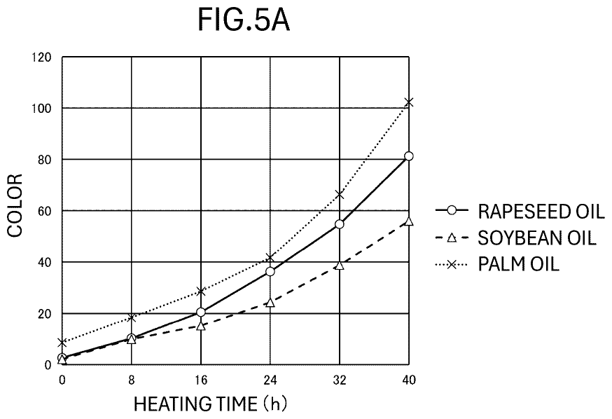

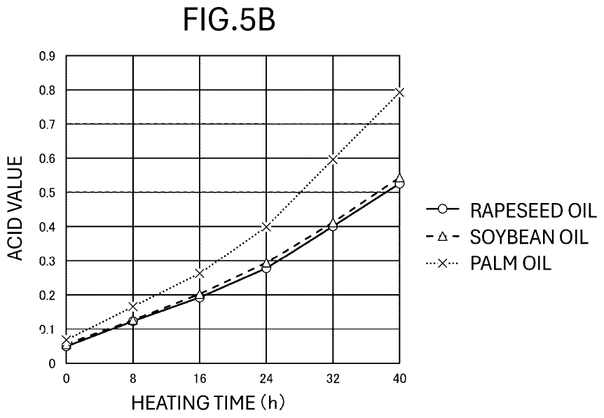

FIG. 5A illustrates a graph showing the correlation between the heating time and color of the frying oil P, for different types of the frying oil P. FIG. 5B illustrates a graph showing the correlation between the heating time and acid value of the frying oil P, for different types of the frying oil P.

FIG. 5A illustrates three patterns of the correlation between the heating time and color of the frying oil P for the cases of the frying oil P of “rapeseed oil”, “soybean oil”, and “palm oil”, respectively. In FIG. 5A, a graph shown with a solid line connecting ∘ marks represents the correlation for the case where the frying oil P is “rapeseed oil”, a graph shown with a broken line connecting A marks represents the correlation for the case where the frying oil P is “soybean oil”, and a graph shown with a dotted line connecting x marks represents the correlation for the case where the frying oil P is “palm oil”, respectively.

FIG. 5B illustrates three patterns of the correlation between the heating time and acid value of the frying oil P for the cases of the frying oil P of “rapeseed oil”, “soybean oil”, and “palm oil”, respectively. In the same manner as FIG. 5A, in FIG. 5B, a graph shown with a solid line connecting ∘ marks represents the correlation for the case where the frying oil P is “rapeseed oil”, a graph shown with a broken line connecting A marks represents the correlation for the case where the frying oil P is “soybean oil”, and a graph shown with a dotted line connecting x marks represents the correlation for the case where the frying oil P is “palm oil”, respectively.

In FIG. 5A illustrating the correlation between the heating time and color of the frying oil P, the frying oil P in the case of rapeseed oil gets deteriorated faster than that in the case of soybean oil, and moreover, the frying oil P in the case of palm oil gets deteriorated faster than that in the case of rapeseed oil.

For example, in FIG. 5A, the heating time until the color value of the frying oil P reaches 40 is about 33 hours in the case where the frying oil P is soybean oil, it is about 26 hours in the case where the frying oil P is rapeseed oil, and it is about 23 hours in the case where the frying oil P is palm oil.

In FIG. 5B illustrating the correlation between the heating time and acid value of the frying oil P, the frying oil P in the case of soybean oil gets deteriorated slightly faster than that in the case of rapeseed oil, and moreover, the frying oil P in the case of palm oil gets deteriorated faster than that in the case of soybean oil.

For example, in FIG. 5B, the heating time until the acid value of the frying oil P reaches 0.3 is about 25 hours in the case where the frying oil P is rapeseed oil, it is about 24 hours in the case where the frying oil P is soybean oil, and it is about 18 hours in the case where the frying oil P is palm oil.

That is, the degradation speed of the frying oil P varies depending on the type of the frying oil P even if the other conditions (the sales of fried foods at a store, the type of the deep-frying material Q, the capacity of the oil vat 21, the amount of fried foods obtained by deep-frying using the frying oil P, and the like) are the same. Therefore, the degradation characteristics (degradation speed) of the frying oil P differ depending on the type of the frying oil P.

In FIG. 2 to FIG. 5, the “correlation between the heating time of the frying oil P and the degradation indicator (specifically, the color and the acid value) of the frying oil P” is used to show the degradation characteristics of the frying oil P, however, any correlation may be used as long as it shows the changes in the degradation indicator, for example, it may be the “correlation between the amount of the deep-frying materials Q (the number of the deep-frying materials Q) that can be deep fried using the frying oil P and the degradation indicator of the frying oil P”.

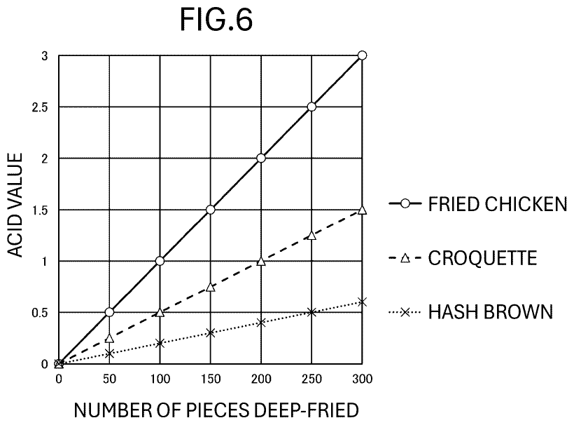

FIG. 6 illustrates a graph showing the correlation between the number of fried foods and acid value of the frying oil P, in the case where the types of the deep-frying materials Q are different from each other.

FIG. 6 illustrates three patterns of the correlation between the heating time and acid value of the frying oil P for the cases where the three types of deep-frying materials Q, which belong in the different categories, are placed in the frying oil P, respectively. In FIG. 6, a graph shown with a solid line connecting ∘ marks represents the correlation for the case of deep-frying fried chickens, a graph shown with a broken line connecting A marks represents the correlation for the case of deep-frying croquettes, and a graph shown with a dotted line connecting x marks represents the correlation for the case of deep-frying hash browns, respectively.

As illustrated in FIG. 6, the slope of the acid value of the frying oil P (corresponding to the degradation speed of the frying oil P) relative the number of croquettes as deep fried is greater than that in the case of deep-frying hash browns, and moreover, the slope of the acid value of the frying oil P in the case of deep-frying fried chickens is greater than that in the case of deep-frying croquettes.

For example, in FIG. 6, the number of materials that can be deep fried until the acid value of the frying oil P reaches 0.5 is 250 in the case of deep-frying hash browns, it is 100 in the case of deep-frying croquettes, and it is 50 in the case of deep-frying fried chickens.

Thus, in this case as well, the degradation characteristics (degradation speed) of the frying oil P vary depending on the type of the deep-frying material Q. The degradation characteristics in the case of deep-frying croquettes show the characteristics in which the frying oil P gets deteriorated more easily than the case of deep-frying hash browns, and moreover, those in the case of deep-frying fried chickens show the characteristics in which the frying oil P gets deteriorated more easily than the case of deep-frying croquettes.

### (Configuration of Degradation Information Processing System 5 for Frying Oil P)

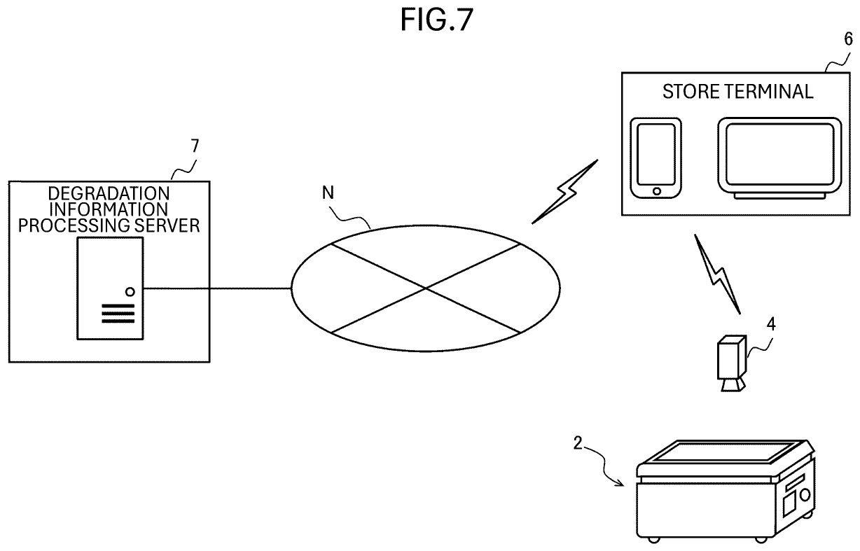

Next, the configuration of a degradation information processing system 5 for the frying oil P will be described with reference to FIG. 7.

FIG. 7 is a system configuration diagram illustrating a configuration example of the degradation information processing system 5 for the frying oil P.

The degradation information processing system 5 for the frying oil P is a system for processing degradation information which is information about the degradation of the frying oil P, and configured with, for example, store terminals 6 installed in a plurality of stores included in a convenience store chain, a supermarket chain, or the like, respectively, and a degradation information processing server 7 configured to execute a program for processing the degradation information about the frying oil P that is used in each store. The store terminal 6 and the degradation information processing server 7 are directly or indirectly connected to each other via a communication network N such as Internet so as to realize the information communication therebetween.

In the degradation information processing system 5 for the frying oil P, the store terminals 6 in the plurality of stores are configured in the same manner from each other, and accordingly, in the following, the store terminal 6 in any of the stores is exemplified while the store terminals 6 in other stores will not be described in detail.

The store terminal 6 is an input terminal to which store information is input, and also a notification device for notifying various kinds of information output from the degradation information processing server 7 (either by displaying texts or outputting sounds). In the store terminal 6, an application for controlling the frying oil P (hereinafter, referred to as a “frying oil control application”) is installed. The store information is information (shop information) about a shop using the frying oil P, and is input by, for example, a staff of the store when initially setting the frying oil control application.

The store information includes customer name information, business type information indicative of the type of business of a store such as a convenience store or a supermarket, fryer information (cooking tool information) which is information about the fryer 2 used in cooking with the frying oil P, oil type information indicative of the type of the frying oil P, and degradation speed information which is information about the degradation speed of the frying oil P.

In the present embodiment, the degradation speed information includes operating hour information indicative of operating hours of the fryer 2 per day, sales information indicative of the sales per day in the store, and number of fryers information indicative of the number of units of the fryer 2 in operation. The sales information is preferably indicative of the sales of the fried food (ingredients cooked using the fryer 2) cooked at the store per day.

In the present embodiment, the store terminal 6 is connected to the camera 4 installed in the cooking area 1 within the store so as to be able to communicate therewith, which allows the store terminal 6 to acquire the data about an image captured by the camera 4 and thus obtain a measured value of the color of the frying oil P within the oil vat 21 of the fryer 2 based on the acquired image. However, the store terminal 6 and the camera 4 do not necessarily have to be connected with each other for communication. In the case where the store terminal 6 and the camera 4 are not connected with each other for communication, the store terminal 6 acquires the data about an image captured by the camera 4 via, for example, an external device.

The degradation information processing server 7 is an aspect of the degradation information processing device for processing the degradation information about the frying oil P. In the following, the degradation information processing server 7 will be described as the one configured with a server device installed in a head office center or the like which manages the plurality of stores as illustrated in FIG. 7, however, it is not necessarily have to be configured with a server device. The degradation information processing server 7 may be configured with, for example, a cloud server or the like constructed on the communication network N.

The degradation information includes at least one of the data indicative of the time remaining until the frying oil P reaches a predetermined oil disposal time point (remaining heating time), the data indicative of the timing at which a removing/adding oil operation is to be performed for the frying oil P in the fryer 2, the data indicative of the timing at which a filtering operation is to be performed for the frying oil P in the fryer 2, and the data indicative of the remaining number of pieces that can be deep-fried until the frying oil P reaches a predetermined degradation degree.

Here, the “removing/adding oil” operation is the operation of removing a part of the frying oil P in the fryer 2 and adding (mixing) frying oil P1 which is different from the frying oil P therein. The “frying oil P1 different from the frying oil P” does not necessarily have to be fresh oil, and may be, for example, frying oil with the degradation degree being less than that of the frying oil P in the fryer 2. Specifically, in the case where three types of dishes, which are tempura, foods covered with bread crumbs, and karaage, are being cooked in the three units of the fryer 2, respectively, in the cooking area 1 at a store, regarding the frying oil P in the oil vat 21 of each fryer 2, the vat for karaage is the most deteriorated, followed by the vat for the foods covered with breaded crumbs, and the vat for tempura is the least deteriorated. In this case, for example, a part of the frying oil P in the vat for tempura or a part of that in the vat for the foods covered with bread crumbs may be mixed into the vat for karaage.

Furthermore, the “filtering” operation is the operation of passing the frying oil P in the fryer 2 through a filter to remove the pieces of foods left in the frying oil P or the like, or passing the frying oil P in the fryer 2 through a material for filtering to clean the frying oil P to make it nearly fresh.

As described above, even if the frying oil P with the degradation degree being progressed reaches the oil disposal time point, it does not necessarily have to be wasted completely or replaced with fresh oil, but the removing/adding oil operation or the filtering operation may be performed therefor before it reaches the oil disposal time point.

Furthermore, the degradation information includes the information about whether the frying oil P in the fryer 2 has reached a degradation criterion value indicative of a criterion relating to the degradation indicator. Here, the degradation criterion value includes at least one of an oil disposal criterion value serving as a criterion for the degradation indicator of the frying oil P at a predetermined oil disposal time point, a removing/adding oil criterion value serving as a criterion for the degradation indicator of the frying oil P in the fryer 2 in the case where a removing/adding oil operation needs to be performed for the frying oil P, and a filtering criterion value serving as a criterion for the degradation indicator of the frying oil P at the time of filtering the frying oil P in the fryer 2.

These degradation criterion values are set in association with at least the customer name information among the information included in the store information. For example, in setting the oil disposal criterion value, the removing/adding oil criterion value, and the filtering criterion value of the frying oil P for the customer “A”, the customer “B”, and the customer “C”, respectively, the values are set to match the specifications of the deep-fry cooking of each customer. However, the degradation criterion values do not necessarily have to be set in association with the store information, but may be, for example, arbitrarily set by a staff of the store or the like.

The degradation information processing server 7 is configured to carry out the degradation prediction processing by predicting at least one of the remaining time until the frying oil P reaches a predetermined oil disposal time point, the timing at which the removing/adding oil operation is to be performed for the frying oil P in the fryer 2, the timing at which the filtering operation is to be performed for the frying oil P in the fryer 2, and the remaining number of pieces that can be deep-fried until the frying oil P reaches a predetermined degradation degree, based on the degradation characteristics of the frying oil P and the degradation criterion value of the frying oil P which are associated with the store information, and outputting the result of prediction to the store terminal 6.

Furthermore, the degradation information processing server 7 is configured to carry out the degradation determination processing by determining at least one of whether the frying oil P has reached the oil disposal time point, whether the frying oil P has reached the removing/adding oil time point, and whether the frying oil P has reached the filtering time point, based on the degradation criterion value associated with the store information, and outputting the result of determination to the store terminal 6.

Still further, the degradation information processing server 7 is configured to calculate, in the degradation determination processing, the amount of removing/adding oil for the frying oil P, and outputs the calculated removing/adding oil amount as the result of calculation of the degradation information about the frying oil P. Here, the “removing/adding oil amount” refers to the amount of the frying oil P in the fryer 2 to be removed (removing oil amount) and the amount of different frying oil P1 to be added into the fryer 2 (adding oil amount) in the removing/adding oil operation, and in typical cases, the removing oil amount and the adding oil amount are the same with each other.

Where a measured value of the degradation indicator of the frying oil P is defined as DI1 (in the case where the measured value of the degradation indicator of the frying oil P is the same as the removing/adding oil criterion value, DI1 is the removing/adding oil criterion value), an after removing/adding oil criterion value, which serves as a criterion for the degradation indicator of the frying oil P after performing the removing/adding oil operation, is defined as DI2, and a degradation indicator of the different frying oil P1 to be added into the fryer 2 is defined as DI3, when the capacity of the oil vat 21 is applied to the remaining oil amount of the frying oil P in the fryer 2 immediately before performing the removing/adding oil operation, the removing/adding oil amount for the frying oil P can be calculated by [Equation 1] below.

$$DI 2 = \frac{\begin{matrix}OIL VAT CAPACITY - \\ REMOVING OIL AMOUNT\end{matrix}}{\begin{matrix}OIL VAT CAPCACITY - REMOVING OIL \\ AMOUNT + ADDING OIL AMOUNT\end{matrix}} \times DI 1 + \frac{ADDING OIL AMOUNT}{\begin{matrix}OIL VAT CAPACITY - REMOVING \\ OIL AMOUNT + ADDING OIL AMOUNT\end{matrix}} \times DI 3 \qquad [ Equation 1 ]$$

In [Equation 1], the capacity of the oil vat 21 is applied to the remaining oil amount of the frying oil P in the fryer 2 immediately before performing the removing/adding oil operation, however, in actual cases, the amount of the frying oil P in the fryer 2 reduces by the amount that the deep-frying material Q absorbs the frying oil P, and thus, it is further preferable to consider the oil absorption amount of the deep-frying material Q, and then calculate the removing/adding oil amount by setting adding oil amount=removing oil amount+oil absorption amount.

Furthermore, the after removing/adding oil criterion value is the value which is set to be a pair with the removing/adding oil criterion value, and is set in association with the customer name information or arbitrarily set by a staff of the store, in the same manner as the removing/adding oil criterion value.

### (Configuration of Degradation Information Processing Server 7)

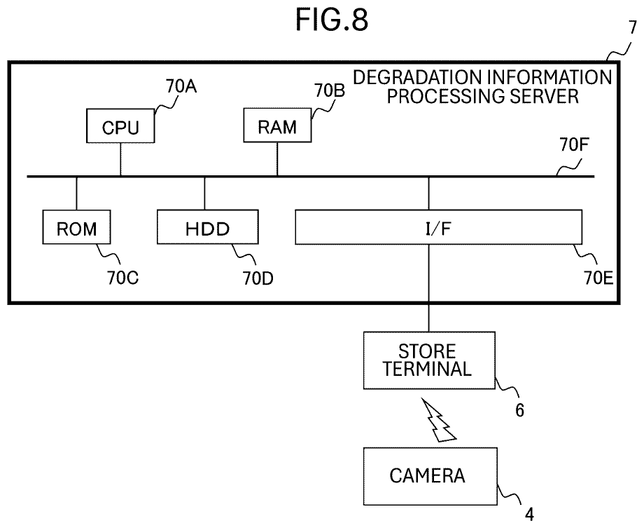

Next, the configuration of the degradation information processing server 7 will be described with reference to FIG. 8 to FIG. 10.

FIG. 8 illustrates an example of a hardware configuration of the degradation information processing server 7.

The degradation information processing server 7 includes a CPU (Central Processing Unit) 70A, a RAM (Random Access Memory) 70B, a ROM (Read Only Memory) 70C, an HDD (Hard Disk Drive) 70D, and an I/F (Interface) 70E, as a hardware configuration of a server device. These components are connected to each other via a common-bus 70F.

The CPU 70A is an arithmetic means and controls the whole operations of the degradation information processing server 7.

The RAM 70B is a volatile storage medium capable of reading and writing information at high speed, and is used, for example, as a working area when the CPU 70A processes image information.

The ROM 70C is a read-only non-volatile storage medium, and retains programs such as firmware.

The HDD 70D is a non-volatile storage medium capable of reading and writing information, and has a large storage capacity in which an OS (Operating System) and control programs and application programs for executing various kinds of information processing, which will be described later, are stored.

Any type of device such as an SSD (Solid State Drive) may be used instead of the HDD 70D as long as it realizes the functions of storing and managing information as a non-volatile storage medium.

The I/F 70E is a connection interface for connection to the communication network N, to which each of the store terminals 6 or the like is connected.

The degradation information processing server 7 including the hardware configuration described above is an information processing device for implementing the processing functions of the control program stored in the ROM 70C, the control program and application program loaded onto the RAM 70B from a storage medium such as the HDD 70D, by means of an arithmetic function provided in the CPU 70A.

By executing the information processing, a software control section including various function modules in the degradation information processing server 7 are implemented. The functional block that realizes the functions of the degradation information processing server 7 is configured with a combination of the software control section thus configured and the hardware resources including the configuration described above.

In the case of the degradation information processing server 7 is configured with a cloud server, the hardware configuration described above is provided in a computer for implementing the cloud server (for example, a computer owned by a company which provides a cloud system or the like).

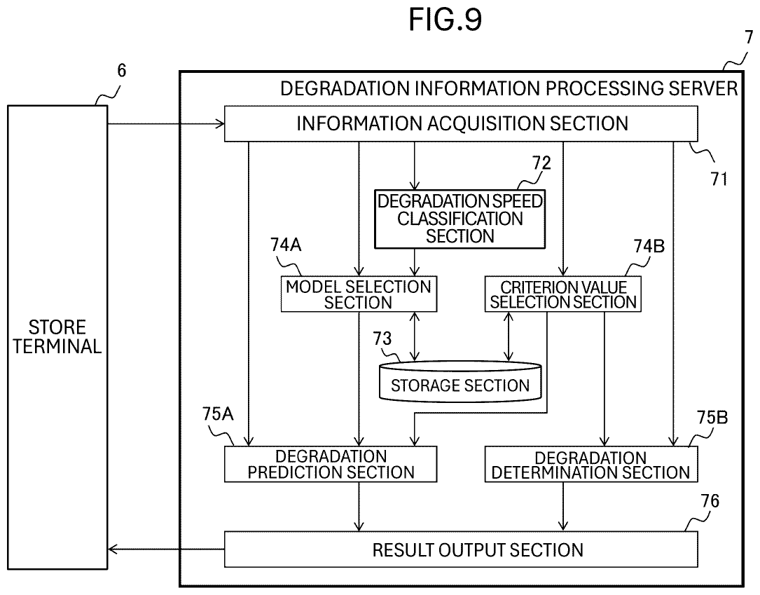

FIG. 9 is a functional block diagram illustrating functions of the degradation information processing server 7.

The degradation information processing server 7 includes an information acquisition section 71, a storage section 73, a degradation speed classification section 72, a model selection section 74A and a criterion value selection section 74B which serve as an information selection section, a degradation prediction section 75A and a degradation determination section 75B which serve as a degradation information processing section, and a result output section 76.

The information acquisition section 71 is configured to acquire the store information and data relating to a measured value of a degradation indicator of the frying oil P, which are output from the store terminal 6, respectively. The information acquisition section 71 acquires, as the store information, in particular, the customer name information, the business type information, the fryer information including the information on the capacity of the fryer 2, the oil type information, the operating hour information, the sales information, and the number of fryers information. However, the information acquisition section 71 does not necessarily have to acquire the operating hour information, the sales information, and the number of fryers information in the case where the store terminal 6 outputs the degradation speed data (specific speed values) on the frying oil P as the degradation speed information.

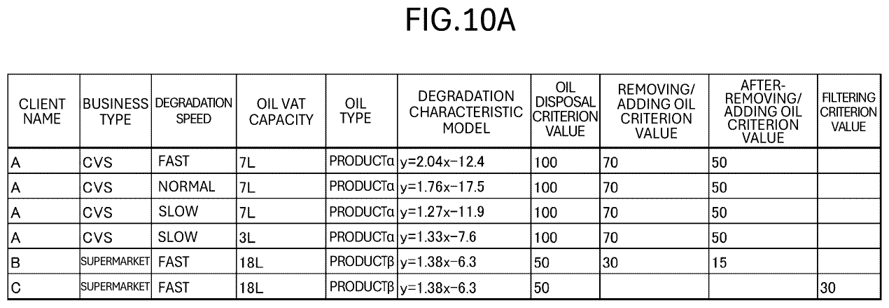

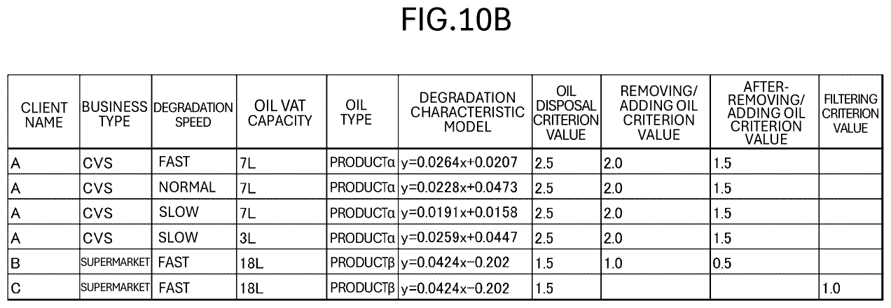

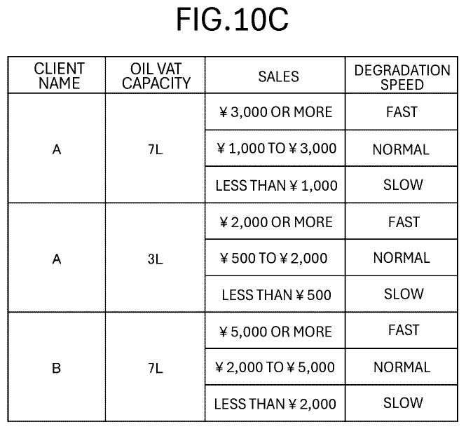

The degradation speed classification section 72 is configured to calculate the sales of one fryer 2 at a store per hour, based on the operating hour information, the sales information, and the number of fryers information acquired by the information acquisition section 71, so as to classify the degradation speed of the frying oil P into one of a plurality of stages (in the present embodiment, three stages of “fast”, “normal”, and “slow” illustrated in FIG. 10A and FIG. 10B).

For example, in the case of a store in which the customer name is “A”, the type of business is “convenience store (CVS)”, the capacity of the oil vat 21 is “7 liters”, and the type of the frying oil P is “product α”, when the operating hours of the fryer 2 per day are “24 hours”, the sales per day are “40,000 yen”, and the number of units of the fryer 2 is “2”, the average sales of one fryer 2 per day are 833 yen (=40,000 yen/2 fryers/24 hours).

Here, the degradation speed in the case where the sales at one unit of the fryer 2 per hour are “equal to or more than 1, 500 yen” is defined as “fast”, the degradation speed in the case where the sales at one unit of the fryer 2 per hour are “equal to or more than 500 yen and less than 1,500 yen” is defined as “normal”, and the degradation speed in the case where the sales at one unit of the fryer 2 per hour are “less than 500 yen” is defined as “slow”. The above-described relation between the classification of the degradation speed of the frying oil P and the sales at one unit of the fryer 2 per hour is shown in a map, and the map is stored in advance in the storage section 73.

In the store described above, the sales at one unit of the fryer 2 per hour are 833 yen, which matches the case of “equal to or more than 500 yen and less than 1,500 yen”, and thus the degradation speed classification section 72 classifies the degradation speed of the frying oil P used in this store into “normal”.

Preferably, the degradation speed classification section 72 is configured to classify the degradation speed of the frying oil P into one of the three stages of “fast”, “normal”, and “slow”, considering at least either of the capacity of the fryer 2 or the customer name information acquired by the information acquisition section 71, relative to the sales at one unit of the fryer 2 per hour at a store as calculated.

FIG. 10C illustrates a map of an example of classification of the degradation speed of the frying oil P.

In FIG. 10C, in a store where the customer name is “A”, the type of business is “convenience store (CVS)”, the capacity of the oil vat 21 of the fryer 2 is “7 liters”, and the type of the frying oil P is “product α”, the degradation speed is classified into “fast” when the sales at one unit of the fryer 2 per hour are “equal to or more than 3,000 yen”, the degradation speed is classified into “normal” when the sales at one unit of the fryer 2 per hour are “equal to or more than 1,000 yen and less than 3,000 yen”, and the degradation speed is classified into “slow” when the sales at one unit of the fryer 2 per hour are “less than 1,000 yen”.

On the other hand, in a store where the customer name is “A”, the type of business is “convenience store (CVS)”, the capacity of the oil vat 21 of the fryer 2 is “3 liters”, and the type of the frying oil P is “product α”, the degradation speed is classified into “fast” when the sales at one unit of the fryer 2 per hour are “equal to or more than 2,000 yen”, the degradation speed is classified into “normal” when the sales at one unit of the fryer 2 per hour are “equal to or more than 500 yen and less than 2,000 yen”, and the degradation speed is classified into “slow” when the sales at one unit of the fryer 2 per hour are “less than 500 yen”.

That is, among the stores with the fryers 2 which are different in their capacities, how the degradation speed of the frying oil P is classified differs even when the customer name, the type of business, and the type of the frying oil P are the same from each other. For example, in the case where the sales at one unit of the fryer 2 per hour are 2,000 yen, the frying oil P gets deteriorated faster when the deep-frying material Q for 2,000 yen are deep-fried with 7 liters of the frying oil P than when the deep-frying material Q for 2,000 yen are deep-fried with 3 liters of the frying oil P. In this case, the degradation speed classification section 72 classifies the degradation speed of the frying oil P into “normal” when the capacity of the oil vat 21 is “7 liters”, while classifying the degradation speed of the frying oil P into “fast” when the capacity of the oil vat 21 is “3 liters”.

Furthermore, in a store where the customer name is “B”, the type of business is “convenience store (CVS),” the capacity of the oil vat 21 of the fryer 2 is “7 liters,” and the type of the frying oil P is “product α”, the degradation speed is classified into “fast” when the sales at one unit of the fryer 2 per hour are “equal to or more than 5,000 yen”, the degradation speed is classified into “normal” when the sales at one unit of the fryer 2 per hour are “equal to or more than 2,000 and less than 5,000 yen,” and the degradation speed is classified into “slow” when the sales at one unit of the fryer 2 per hour are “less than 2,000 yen”.

Thus, among the stores with the different customer names (different companies), how the degradation speed of the frying oil P is classified differs depending on the type of the deep-frying material Q to be cooked, the size of company, the type of target customers, its popularity, and the like even when the type of business, the capacity of the fryer 2, and the type of the frying oil P are the same from each other. Specifically, even when the sales at one unit of the fryer 2 per hour are the same, that is, even when the degradation speed of the frying oil P itself is the same between the customer “A” and the customer “B”, if the customer “A” and the customer “B” differ in their company size, how the degradation speed of the frying oil P is classified differs relatively therebetween. For example, in the case where the company size of the customer “B” is larger than that of the customer “A”, the degradation speed classification section 72 classifies the degradation speed of the frying oil P used by the customer “A” into “normal”, while classifying the degradation speed of the frying oil P used by the customer “B” into “slow”.

In the case where the information acquisition section 71 has acquired the degradation speed data about the frying oil P, the degradation speed classification section 72 classifies the degradation speed of the frying oil P into one of a plurality of stages based on the degradation speed of the frying oil P per se as acquired.

The storage section 73 retains, in addition to the map showing the relation between the classification of the degradation speed of the frying oil P and the sales per hour at one unit of the fryer 2, a plurality of pieces of degradation indicator related information which is the information about a degradation indicator of the frying oil P associated with the store information. The “degradation indicator related information” includes the data about a degradation characteristic model of the frying oil P which is indicative of the change in the degradation indicator and the data about various degradation criterion values of the frying oil P.

FIG. 10A illustrates a degradation indicator related map stored in the storage section 73, which is an example using the color of the frying oil P as a degradation indicator. FIG. 10B illustrates a degradation indicator related map stored in the storage section 73, which is an example using the acid value of the frying oil P as a degradation indicator. In the present embodiment, the degradation indicator related map for the color of the frying oil P and the degradation indicator related map for the acid value of the frying oil P are described with reference to FIG. 10A and FIG. 10B, however, for other degradation indicators, degradation indicator related maps can be created in the same manner as the degradation indicator related maps illustrated in FIG. 10A and FIG. 10B.

As illustrated in FIG. 10A and FIG. 10B, the storage section 73 retains the plurality of degradation characteristic models of the frying oil P and the plurality of the oil disposal criterion values of the frying oil P, removing/adding oil criterion values of the frying oil P, after-removing/adding oil criterion values of the frying oil P, and filtering criterion values of the frying oil P. They are associated with the store information, respectively, and make up the degradation indicator related map.

For example, the store information with “A” as the customer name, “convenience store (CVS)” as the business type, “fast” as the degradation speed of the frying oil P, “7 liters” as the capacity of the oil vat 21, and “product α” as the type of the frying oil P is associated with “color value; y=2.04x−12.4” or “acid value; y=0.0264x+0.0207” as the degradation characteristic model, “color value=100” or “acid value=2.5” as the oil disposal criterion value, “color value=70” or “acid value=2.0” as the removing/adding oil criterion value, and “color value=50” or “acid value=1.5” as the after-removing/adding oil criterion value, respectively. In this store, for the frying oil P, the removing/adding oil operation is performed but a filtering operation is not performed, and accordingly, the filtering criterion value is not set.

Furthermore, for example, the store information with “B” as the customer name, “supermarket” as the business type, “fast” as the degradation speed of the frying oil P, “18 liters” as the capacity of the oil vat 21, and “product β” as the type of the frying oil P is associated with “color value; y=1.38x−6.3” or “acid value; y=0.0424x−0.202” as the degradation characteristic model, “color value=50” or “acid value=1.5” as the oil disposal criterion value, “color value=30” or “acid value=1.0” as the removing/adding oil criterion value, and “color value=15” or “acid value=0.5” as the after-removing/adding oil criterion value, respectively. Also in this store, for the frying oil P, the removing/adding oil operation is performed but the filtering operation is not performed, and accordingly, the filtering criterion value is not set.

Still further, for example, the store information with “C” as the customer name, “supermarket” as the business type, “fast” as the degradation speed of the frying oil P, “18 liters” as the capacity of the oil vat 21, and “product β” as the type of the frying oil P is associated with “color value; y=1.38x−6.3” or “acid value; y=0.0424x−0.202” as the degradation characteristic model, “color value=50” or “acid value=1.5” as the oil disposal criterion value, “color value=30” or “acid value=1.0” as the filtering criterion value, respectively. In this store, for the frying oil P, the filtering operation is performed but the removing/adding oil operation is not performed, and accordingly, neither the removing/adding oil criterion value nor the after-removing/adding oil criterion value is not set.

The value “x” in each of the degradation characteristic models listed in FIG. 10A and FIG. 10B corresponds to the horizontal axis of each of FIG. 2 to FIG. 5, that is, the “heating time of the frying oil P”, and the value “y” corresponds to the vertical axis of each of FIG. 2 to FIG. 5, that is, the “degradation indicator of the frying oil P”.

In FIG. 10A and FIG. 10B, a degradation characteristic model is exemplified by the model showing the correlation between the heating time of the frying oil P and the degradation indicator of the frying oil P, however, it is not limited thereto. The degradation characteristic model also includes a model showing the correlation between the number of the deep-frying materials Q that can be deep-fried with the frying oil P and the degradation indicator of the frying oil P (model corresponding to the graph illustrated in FIG. 6).

The model showing the correlation between the number of the deep-frying materials Q that can be deep-fried with the frying oil P and the degradation indicator of the frying oil P is expressed by, for example, [Equation 2] below.

$$Δ AV = 0.01 \times α + 0.005 \times β + 0.002 \times γ \qquad [ Equation 2 ]$$

In [Equation 2], AAV represents a difference between the oil disposal criterion value of the frying oil P and a measured value of the degradation indicator of the frying oil P. Furthermore, α, β, and γ represent the number of the deep-frying materials Q that can be deep-fried, respectively.

The easiness of getting dark in color, increase degree of viscosity, influence on the acid value, and the like differ depending on the type of the deep-frying material Q, and thus the amount of variation in the degradation indicator of the frying oil P differs depending on the type of the deep-frying material Q. For example, where α is defined as the number of fried chickens to be deep-fried, β is defined as the number of croquettes to be deep-fried, and γ is the number of hash browns to be deep-fried, the influences of the deep-frying materials Q on the degradation indicator are “0.01” for fried chickens, “0.005” for croquettes, and “0.002” for hash browns, respectively.

For example, in the case of setting the oil disposal criterion value of the frying oil P to “acid value 2.5”, when a measured value of the degradation indicator of the frying oil P is “acid value 1.5”, ΔAV is “1.0”.

In the case of deep-frying only fried chickens using the frying oil P, by substituting “1.0” into ΔAV and “0” into β and γ, respectively, in [Equation 2], α is obtained as “100”. Thus, it can be predicted that the remaining number of fried chickens that can be deep-fried until the frying oil P reaches the oil disposal criterion value is 100.

In the case of deep-frying only croquettes using the frying oil P, by substituting “1.0” into ΔAV and “0” into α and γ, respectively, in [Equation 2], β is obtained as “200”. Thus, it can be predicted that the remaining number of croquettes that can be deep-fried until the frying oil P reaches the oil disposal criterion value is 200.

In the case of deep-frying only hash browns using the frying oil P, by substituting “1.0” into ΔAV and “0” into α and β, respectively, in [Equation 2], γ is obtained as “500”. Thus, it can be predicted that the remaining number of hash browns that can be deep-fried until the frying oil P reaches the oil disposal criterion value is 500.

In the case of deep-frying fried chickens, croquettes, and hash browns on average using the frying oil P, by substituting “1.0” into ΔAV and setting α=β=γ in [Equation 2], α, β, and γ are obtained as “58”, respectively. Thus, it can be predicted that the remaining number of fried chickens, croquettes, and hash browns that can be deep-fried until the frying oil P reaches the oil disposal criterion value is 58, respectively.

In the case of specifying 50 pieces of fried chickens to be deep-fried using the frying oil P and deep-frying only croquettes for the remaining pieces, by substituting “1.0” into ΔAV, “50” into α, and “0” into γ, respectively, in [Equation 2], β is obtained as “100”. Thus, it can be predicted that the number of croquettes that can be deep-fried until the frying oil P reaches the oil disposal criterion value is 100.

In the case of specifying 50 pieces of fried chickens to be deep-fried using the frying oil P and deep-frying only hash browns for the remaining pieces, by substituting “1.0” into ΔAV, “50” into α, and “0” into β, respectively, in [Equation 2], γ is obtained as “250”. Thus, it can be predicted that the number of hash browns that can be deep-fried until the frying oil P reaches the oil disposal criterion value is 250.

In the case of specifying 50 pieces of fried chickens to be deep-fried using the frying oil P and deep-frying croquettes and hash browns equally for the remaining pieces, by substituting “1.0” into ΔAV, “50” into α, respectively, and setting β=γ in [Equation 2], β and γ are obtained as “71”, respectively. Thus, it can be predicted that the remaining number of croquettes and hash browns that can be deep-fried until the frying oil P reaches the oil disposal criterion value is 71, respectively.

In the examples described above, the number of pieces that can be deep-fried until the frying oil P reaches the oil disposal criterion value is predicted, however, the prediction is not limited thereto. Prediction using the same degradation characteristic model can be carried out for the number of pieces that can be deep-fried until the frying oil P reaches a predetermined degradation degree (removing/adding oil criterion value or filtering criterion value).

As described above, the degradation characteristic model includes at least one of a model showing the correlation between the heating time of the frying oil P and the degradation indicator of the frying oil P, and a model showing the correlation between the number of deep-frying materials Q that can be deep-fried with the frying oil P and the degradation indicator of the frying oil P. Thus, the degradation characteristic model corresponds to each of the graphs showing the degradation characteristics of the frying oil P illustrated in FIG. 2 to FIG. 6, and using the degradation characteristic model enables prediction of the degradation of the frying oil P. Accordingly, it can be also said that the degradation characteristic model serves as a “degradation prediction model”.

Although each of the degradation characteristic models illustrated in FIG. 10A and FIG. 10B are the model of single regression expressed in the form of linear equation such as y=ax+b (a: slope, b: intercept), the type of degradation characteristic model is not particularly limited, and, for example, a model of linear regression other than single regression such as multiple regression, model generated by machine learning, and the like may be used.

In the degradation indicator related map illustrated in each of FIG. 10A and FIG. 10B, for the store where the removing/adding oil criterion value and the after-removing/adding oil criterion value are set, the filtering criterion value is not set, and for the store where the filtering criterion value is set, the removing/adding oil criterion value and the after-removing/adding oil criterion value are not set, however, the degradation indicator related map is not limited thereto. The degradation indicator related map may include a store where all the items of the removing/adding oil criterion value and the after-removing/adding oil criterion value, and the filtering criterion value are set may be included, or a store where neither the removing/adding oil criterion value and the after-removing/adding oil criterion value or the filtering criterion value is set.

Furthermore, the degradation indicator related map illustrated in each of FIG. 10A and FIG. 10B includes the degradation characteristic model of the frying oil P and various degradation criterion values of the frying oil P (oil disposal criterion value of the frying oil P, removing/adding oil criterion value of the frying oil P, after-removing/adding oil criterion value of the frying oil P, and filtering criterion value of the frying oil P), however, the degradation indicator related map does not necessarily have to include both of them. The degradation information processing server 7 may store a degradation indicator related map which depends on the content to be processed by the degradation information processing server 7 (that is, whether the degradation prediction processing for the frying oil P is to be carried out or the degradation determination processing for the frying oil P is to be carried out).

The model selection section 74A illustrated in FIG. 9 is configured to select one of the degradation characteristic models (six in both FIG. 10A and FIG. 10B) stored in the storage section 73, which corresponds to the store information acquired by the information acquisition section 71.

More specifically, the model selection section 74A selects one of the degradation characteristic models stored in the storage section 73, based on the business type information, the fryer information, and the oil type information acquired by the information acquisition section 71, and the stage classified by the degradation speed classification section 72.

For example, in the case where the information acquisition section 71 acquires the store information with “C” as the customer name, “supermarket” as the business type, “18 liters” as the capacity of the oil vat 21, and “product β” as the type of the frying oil P, and also the degradation speed classification section 72 classifies the degradation speed of the frying oil P as “fast”, the model selection section 74A selects the degradation characteristic model of “color value; y=1.38x−6.3” listed in the lowest row of FIG. 10A for the case using the color as the degradation indicator, and selects the degradation characteristic model of “acid value; y=0.0424x−0.202” listed in the lowest row of FIG. 10B for the case using the acid value as the degradation indicator.

The criterion value selection section 74B is configured to select degradation criterion values corresponding to the store information acquired by the information acquisition section 71, from among a plurality of various degradation criterion values (six in both FIG. 10A and FIG. 10B) stored in the storage section 73.

For example, when the degradation information processing server 7 determines whether to dispose the frying oil P or predicts when to dispose the frying oil P by, in the case where the information acquisition section 71 acquires the store information with “C” as the customer name, “supermarket” as the business type, “fast” as the degradation speed of the frying oil P, “18 liters” as the capacity of the oil vat 21, and “product β” as the type of the frying oil P, the criterion value selection section 74B selects the oil disposal criterion value “color value=50” listed in the lowest row of FIG. 10A for the case using the color as the degradation indicator, and selects the oil disposal criterion value “acid value=1.5” listed in the lowest row of FIG. 10B for the case using the acid value as the degradation indicator.

Furthermore, for example, when the degradation information processing server 7 determines whether to remove and add oil for the frying oil P or predicts when to remove and add oil for the frying oil P, in the case where the information acquisition section 71 acquires the store information with “B” as the customer name, “supermarket” as the business type, “fast” as the degradation speed of the frying oil P, “18 liters” as the capacity of the oil vat 21, and “product β” as the type of the frying oil P, the criterion value selection section 74B selects the oil disposal criterion value “color value=30” listed in the second row from the bottom of FIG. 10A for the case using the color as the degradation indicator, and selects the oil disposal criterion value “acid value=1.0” listed in the second row from the bottom of FIG. 10B for the case using the acid value as the degradation indicator.

Based on the measured value of the degradation indicator of the frying oil P acquired by the information acquisition section 71, the degradation characteristic model selected by the model selection section 74A, and the degradation criterion value selected by the criterion value selection section 74B, the degradation prediction section 75A predicts at least one of the remaining heating time until the frying oil P reaches a predetermined oil disposal time point, the timing at which the removing/adding oil operation is to be performed for the frying oil P in the fryer 2, the timing at which the filtering operation is to be performed for the frying oil P in the fryer 2, and the remaining number of pieces that can be deep-fried until the frying oil P reaches a predetermined degradation degree.

Specifically, the degradation prediction section 75A predicts the remaining heating time until the frying oil P reaches a predetermined oil disposal time point, according to the following procedure.

For example, in a store where the customer name is “A”, the type of business is “convenience store (CVS)”, the degradation speed of the frying oil P is “fast”, the capacity of the oil vat 21 is “7 liters”, and the type of the frying oil P is “product α”, when the degradation indicator is the acid value, the model selection section 74A selects the degradation characteristic model of y=0.0264x+0.0207, and the criterion value selection section 74B selects the oil disposal criterion value 2.5 (see degradation indicator related map illustrated in FIG. 10B).

Here, when a measured value of the acid value of the frying oil P at a certain time point is 1.0, the degradation prediction section 75A calculates that the heating time (=x) of the frying oil P is 37 hours by substituting 1.0 into y in the degradation characteristic model. Furthermore, the degradation predictor 75A calculates that the heating time x of the frying oil P at the oil disposal criterion value of 2.5 is 94 hours by substituting the oil disposal criterion value of 2.5 into y of the degradation characteristic model. Then, the degradation prediction section 75A subtracts the heating time (=37 hours) of the frying oil P at the certain time point (at the time point of the measured acid value 1.0) from the heating time of the frying oil P (=94 hours) at the oil disposal criterion value of 2.5 to predict that the remaining heating time until the frying oil P reaches a predetermined oil disposal time point is 57 hours (=94 hours−37 hours).

That is, substituting a measured value of the degradation indicator of the frying oil P acquired by the information acquisition section 71 and the degradation criterion value selected by the criterion value selection section 74B into the degradation characteristic model which was selected by the model selection section 74A enables prediction of the remaining heating time until the frying oil P reaches a predetermined oil disposal time point.

This prediction procedure also applies to the prediction of the timing at which the removing/adding oil operation is to be performed for the frying oil P in the fryer 2, the timing at which the filtering operation is to be performed for the frying oil P in the fryer 2, and the remaining number of pieces that can be deep-fried until the frying oil P reaches a predetermined degradation degree.

Based on the measured value of the degradation indicator of the frying oil P acquired by the information acquisition section 71 and the degradation criterion value which was selected by the criterion value selection section 74B, the degradation determination section 75B determines at least one of whether the frying oil P has reached the oil disposal time point, whether the frying oil P has reached the removal/adding oil time point, and whether the frying oil P has reached the filtering time point.

Furthermore, in the present embodiment, the degradation determination section 75B calculates the removing/adding oil amount for the frying oil P, based on the fryer information (specifically, data related to the capacity of the oil vat 21) and the measured value of the degradation indicator of the frying oil P, which were acquired by the information acquisition section 71, and the after-removing/adding oil criterion value selected by the criterion value selection section 74B. For accurately calculating the removing/adding oil amount for the frying oil P by the degradation determination section 75B, the information acquisition section 71 acquires, from the store terminal 6, the data related to the oil absorption amount, which is the amount of the frying oil P that the deep-frying material Q has absorbed.

The result output section 76 is configured to output the result of prediction carried out by the degradation prediction section 75A and the result of determination carried out by the degradation determination section 75B to the store terminal 6. The output section 76 also outputs, if any, the removing/adding oil amount for the frying oil P calculated by the degradation determination section 75B to the store terminal 6.

The degradation information processing server 7 does not necessarily have to include the model selection section 74A and the degradation prediction section 75A, which function to predict the degradation of the frying oil P, and the criterion value selection section 74B and the degradation determination section 75B, which function to determine the degradation of the frying oil P, as long as it includes at least one of the degradation prediction function for the frying oil P and the degradation determination function fir the frying oil P. In the case where the degradation information processing server 7 has only the degradation determination function for frying oil, the degradation information processing server 7 does not necessarily have to include the degradation speed classification section 72.

### (Processing to be Executed in Degradation Information Processing Server 7)

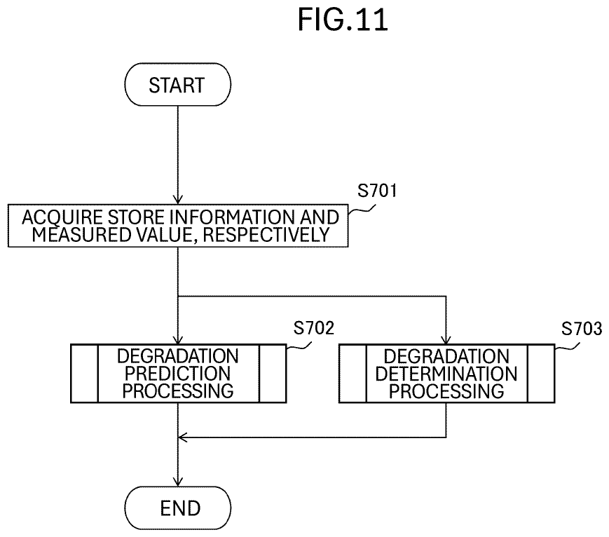

Next, the processing to be executed in the degradation information processing server 7 will be described with reference to FIG. 11 to FIG. 13.

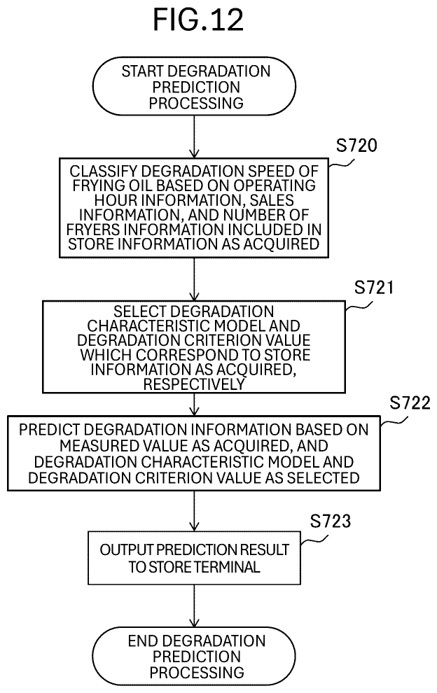

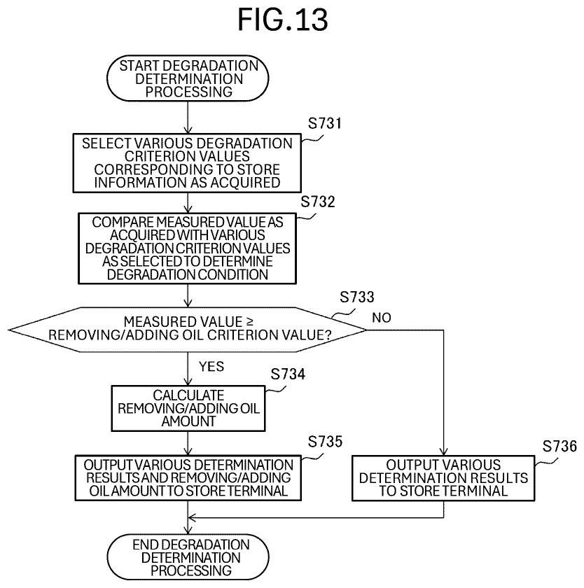

FIG. 11 illustrates a flowchart of an overall flow of the processing to be executed by the degradation information processing server 7. FIG. 12 illustrates a flowchart of a flow of the degradation prediction processing. FIG. 13 illustrates a flowchart of an example of a flow of the degradation determination processing.

As illustrated in FIG. 11, in the degradation information processing server 7, firstly, the information acquisition section 71 acquires the store information and the measured value of the degradation indicator of the frying oil P, which are output from the store terminal 6, respectively (step S701; information acquiring step).

Next, the degradation information processing server 7 proceeds to the degradation prediction processing (step S702) and the degradation determination processing (step S703). In the case of having only the degradation prediction function, the degradation information processing server 7 proceeds to the degradation prediction processing (step S702), and in the case of having only the degradation determination function, the degradation information processing server 7 proceeds to the degradation determination processing (step S703).

In the degradation prediction processing (step S702), as illustrated in FIG. 12, firstly, the degradation speed classification section 72 classifies the degradation speed of the frying oil P into one of “slow”, “normal”, and “fast” stages based on the operating hour information, the sales information, and the number of fryers information included in the store information acquired in step S701 (step S720; degradation speed classifying step).

Next, the model selection section 74A selects a characteristic model corresponding to the store information acquired in step S701 from among the plurality of degradation characteristic models stored in the storage section 73, based on the business type information, the fryer information, and the oil type information included in the store information acquired in step S720, and the stage of the degradation speed which was classified in step S720. Also, the criterion value selection section 74B selects a degradation criterion value corresponding to the store information (customer name information) acquired in step S701 from among the plurality of various degradation criterion values stored in the storage section 73 (step S721; information selection step).

Next, the degradation prediction section 75A predicts the degradation information about the frying oil P based on the measured value of the degradation indicator of the frying oil P acquired in step S701 and the degradation characteristic model and the degradation criterion value which were selected in step S721 (step S722; degradation information processing step).

Then, the result output section 76 outputs the result of prediction obtained in step S722 to the store terminal 6 (step S723; processing result outputting step), whereby the processing in the degradation information processing server 7 ends. Upon receiving the result of prediction from the result output section 76 (degradation information processing server 7), the store terminal 6 notifies a staff of the store of the information as received (for example, “XX hours left until the oil is to be disposed” or “please perform the removing/adding oil operation after XX hours”), by means of a screen display, voices, or the like (notifying step).

In the degradation determination processing (step S703), as illustrated in FIG. 13, firstly, the criterion value selection section 74B selects various degradation criterion values, which correspond to the store information acquired in step S701, from among the plurality of various degradation criterion values stored in the storage section 73 (step S731; information selecting step).

Next, the degradation determination section 75B compares the measured value of the degradation indicator of the frying oil P acquired in step S701 with the various degradation criterion values which were selected in step S731 to determine the degradation condition (degree of degradation) of the frying oil P (step S732; information processing step).

Upon determining that the measured value of the degradation indicator of the frying oil P acquired in step S701 is equal to or more than the removing/adding oil criterion value (step S733/YES), the degradation determination section 75B calculates the removing/adding oil amount for the frying oil P (step S734; information processing step).

In step S734, the degradation determination section 75B uses [Equation 1] described above to calculate the removing/adding oil amount necessary in the removing/adding oil operation for the frying oil P, based on the measured value of the degradation indicator of the frying oil P and the remaining oil amount data about the frying oil P acquired by the information acquisition section 71, and the after-removing/adding oil criterion value selected by the criterion value selection section 74B. Thus, in the case where the degradation determination section 75B calculates the removing/adding oil amount for the frying oil P, the remaining oil amount data of the frying oil P is also acquired in step S701, and the after-removing/adding oil criterion value is also selected in step S731.

Then, the result output section 76 outputs, to the store terminal 6, the various determination results obtained in step S732 and the removing/adding oil amount for the frying oil P calculated in step S734 as the result of calculation of the degradation information about the frying oil P (step S735; processing result outputting step), whereby the processing in the degradation information processing server 7 ends. Upon receiving the various determination results and the removing/adding oil amount for the frying oil P from the result output section 76 (degradation information processing server 7), the store terminal 6 notifies a staff of the store of the information as received (for example, “please perform the removing/adding oil operation for the frying oil P, by the amount of XX liters”), by means of a screen display, voices, or the like (notifying step).

On the other hand, in step S733, when it is not determined that the measured value of the degradation indicator of the frying oil P is equal to or more than the removing/adding oil criterion value (including the case where the measured value of the degradation indicator of the frying oil P is not compared with the removing/adding oil criterion value) (step S733/NO), the result output section 76 outputs the various determination results obtained in step S732 to the store terminal 6 (step S736), whereby the processing in the degradation information processing server 7 ends. Upon receiving the various determination results from the result output section 76 (degradation information processing server 7), the store terminal 6 notifies a staff of the store of the information as received (for example, “the removing/adding oil operation does not have to be performed for the frying oil P now”), by means of a screen display, voices, or the like (notifying step).

In step S732, even when it is determined that the measured value of the degradation indicator of the frying oil P is equal to or more than the removing/adding oil criterion value (step S733/YES), the removing/adding oil amount for the frying oil P does not necessarily have to be calculated (step S734). In such a case, it may be configured that the result output section 76 outputs only the various determination results obtained in step S732 to the store terminal 6, and the store terminal 6 provides notification, such as “please dispose the frying oil P”.

Furthermore, in the degradation determination processing (step S703), it may be configured to calculate only the removing/adding oil amount for the frying oil P (step S734), so that the result output section 76 outputs the removing/adding oil amount for the frying oil P calculated in step S734 to the store terminal 6.

Still further, the degradation information processing server 7 does not necessarily have to execute both the degradation prediction processing (step S702) and the degradation determination processing (step S703), and may execute only either of them (step S702 or step S703).

As described above, the degradation information processing server 7 is configured to carry out the degradation prediction processing for the frying oil P based on the degradation characteristic model (degradation indicator related information) associated with the store information which is specific to each store using the frying oil P, and the measurement value of the degradation indicator of the frying oil P. This enables the result of prediction to be obtained with higher accuracy than in the case of carrying out the degradation prediction processing for the frying oil P using a degradation characteristic model set for all cases without considering the store information.

This allows a store using the frying oil P to avoid the possibilities that the frying oil P that can still be used is wasted or the frying oil P that has already passed the time for disposal is still be used. Accordingly, the present invention can contribute to the efforts to promote the Sustainable Development Goals (2030 Agenda for Sustainable Development, adopted by the United Nations on Sep. 25, 2015, hereinafter, referred as “SDGs”).

Furthermore, in the degradation information processing server 7 according to the present embodiment, the degradation speed classification section 72 is configured to classify the degradation speed of the frying oil P into one of the plurality of stages based on the operating hour information, the sales information, and the number of fryers information acquired by the information acquisition section 71. This enables the degradation speed of the frying oil P to be classified into one of the plurality of stages with less time and effort, as compared with the case of measuring the degradation speed of the frying oil P using various measuring instruments to perform classification.

In the above, the embodiment of the present invention has been described. However, the present invention is not limited to the embodiment described above but various modifications can be made therein. For example, the embodiment is described in detail herein for the purpose of clarity and concise description, and the present invention is not limited to those including all the features described above. Furthermore, some of the features according to a predetermined embodiment can be replaced with other features according to a separate embodiment, and other features can be added to the configuration of the predetermined embodiment. Still further, other features of a separate embodiment may be added to some of the features of each of the embodiments described above, and some of the features of each of the embodiments described above may be deleted or replaced.

For example, in the embodiment described above, the degradation information processing server 7 has been described as an aspect of the degradation information processing device for the frying oil P, however, the functions of the degradation information processing device for the frying oil P may be provided in a frying oil management application installed in the store terminal 6. In this case, the store terminal 6 serves not only as an input terminal and a notification device, but also as a degradation information processing device for the frying oil P.

Furthermore, in the embodiment described above, the degradation indicator of the frying oil P has been mainly described referring to the cases where it is the color or the acid value, however, the degradation indicator of the frying oil P is not limited thereto. The present invention can be realized even if using a degradation indicator other than the color or the acid value.

### REFERENCE SIGNS LIST

5: degradation information processing system
6: store terminal (input terminal, notification device, degradation information processing device)
7: degradation information processing server (degradation information processing device)
71: information acquisition section
72: degradation speed classification section
73: storage section
74A: model selection section (information selection section)
74B: criterion value selection section (information selection section)
75A: degradation prediction section (degradation information processing section)
75B: degradation determination section (degradation information processing section)
76: result output section
P: frying oil (edible oil)
Q: deep-frying material (ingredient)

1. An edible oil degradation information processing device comprising:
a storage section configured to retain a plurality of pieces of degradation indicator related information which is information about a degradation indicator of an edible oil, the degradation indicator related information being associated with store information which is information about a store using the edible oil;
an information acquisition section configured to acquire the store information;
an information selection section configured to select the degradation indicator related information corresponding to the store information acquired by the information acquisition section, from among the plurality of pieces of degradation indicator related information stored in the storage section;
a degradation information processing section configured to process degradation information which is information about degradation of the edible oil, based on the degradation indicator related information selected by the information selection section; and
a result output section configured to output a processing result of the degradation information processed by the degradation information processing section.

2. The edible oil degradation information processing device according to claim 1, wherein
the store information includes:
business type information indicative of a type of business of the store;
cooking tool information which is information about a cooking tool used for cooking with the edible oil;
oil type information indicative of a type of the edible oil; and
degradation speed information which is information about a degradation speed of the edible oil.

3. The edible oil degradation information processing device according to claim 2, wherein
the degradation speed information includes:
operating hour information indicative of operating hours of the cooking tool in a day;
sales information indicative of sale of the store in a day; and
number of tools information indicative of the number of units of the cooking tool in operation.

4. The edible oil degradation information processing device according to claim 1, wherein
the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively,
the information acquisition section is configured to further acquire data about a measured value of the degradation indicator,
the information selection section is configured to:
select one of the degradation characteristic models stored in the storage section, which corresponds to the store information acquired by the information acquisition section; and
select one of the degradation criterion values stored in the storage section, which corresponds to the store information acquired by the information acquisition section;
the degradation information processing section is configured to predict the degradation information based on the measured value of the degradation indicator acquired by the information acquisition section, and the one of the degradation characteristic models and the one of the degradation criterion values selected by the information selection section, and
the result output section is configured to output a prediction result of the degradation information obtained by the degradation information processing section.

5. The edible oil degradation information processing device according to claim 4, wherein
the degradation information includes at least one of data indicative of a remaining time until the edible oil reaches a predetermined oil disposal time point, data indicative of a timing of removing a part of the edible oil in a cooking tool used for cooking with the edible oil and adding another edible oil, which is different from the edible oil, in the edible oil, data indicative of a timing of filtering the edible oil in the cooking tool, or data indicative of an amount of remaining ingredients that can be cooked until the edible oil reaches a predetermined degradation degree.

6. The edible oil degradation information processing device according to claim 4, wherein
the degradation characteristic model includes at least one of a model indicative of a correlation between a heating time of the edible oil and the degradation indicator, or a model indicative of a correlation between an amount of ingredients that can be cooked with the edible oil and the degradation indicator.

7. The edible oil degradation information processing device according to claim 4, further comprising a degradation speed classification section configured to classify a degradation speed of the edible oil, wherein
the information acquisition section is configured to acquire, as the store information, business type information indicative of a type of business of the store, cooking tool information which is information about a cooking tool used for cooking with the edible oil, oil type information indicative of a type of the edible oil, operating hour information indicative of operating hours of the cooking tool in a day, sales information indicative of sale of the store in a day, and number of tools information indicative of the number of units of the cooking tool in operation,
the degradation speed classification section is configured to, based on the operating hour information, the sales information, and the number of tools information acquired by the information acquisition section, calculate sales at one unit of the cooking tool per hour in the store to classify the degradation speed of the edible oil into one of a plurality of stages, and
the information selection section is configured to, based on the business type information, the cooking tool information, and the oil type information acquired by the information acquisition section, and the one of the plurality of stages classified by the degradation speed classification section, select one of the degradation characteristic models, which corresponds to the store information.

8. The edible oil degradation information processing device according to claim 7, wherein
the information acquisition section is configured to further acquire, as the store information, customer name information about the store, and
the degradation speed classification section is configured to classify the degradation speed of the edible oil into one of the plurality of stages, considering, relative to the sales at one unit of the cooking tool per hour in the store as calculate, at least either a capacity of the cooking tool included in the cooking tool information acquired by the information acquisition section or the customer name information.

9. The edible oil degradation information processing device according to claim 1, wherein
the plurality of pieces of degradation indicator related information includes data about degradation criterion values, each of which is indicative of a criterion relating to the degradation indicator, respectively,
the information acquisition section is configured to further acquire data about a measured value of the degradation indicator,
the information selection section is configured to select one of the degradation criterion values stored in the storage section, which corresponds to the store information acquired by the information acquisition section,
the degradation information processing section is configured to, based on the measured value of the degradation indicator acquired by the information acquisition section and the one of the degradation criterion values selected by the information selection section, determine whether the edible oil has reached the one of the degradation criterion values, and
the result output section is configured to output, as a determination result of the degradation information, a result determined by the degradation information processing section.

10. The edible oil degradation information processing device according to claim 9, wherein
the degradation criterion values include at least one of an oil disposal criterion value serving as a criterion for the degradation indicator at a predetermined oil disposal time point, a removing/adding oil criteria value serving as a criterion for the degradation indicator in a case where a removing/adding oil operation of removing a part of the edible oil in the cooking tool and adding another edible oil, which is different from the edible oil, in the edible oil needs to be performed, or a filtering criterion value serving as a criterion for the degradation indicator at a time of filtering the edible oil in the cooking tool.

11. The edible oil degradation information processing device according to claim 1, wherein
the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil,
the information acquisition section is configured to further acquire data about a measured value of the degradation indicator and remaining oil amount data which is data about a remaining oil amount of the edible oil in the cooking tool immediately before performing the removing/adding oil operation,
the information selection section is configured to select one of the after-removing/adding oil criterion values stored in the storage section, which corresponds to the store information acquired by the information acquisition section,
the degradation information processing section is configured to calculate a removing/adding oil amount necessary in the removing/adding oil operation, based on the measured value of the degradation indicator and the remaining oil amount data acquired by the information acquisition section, and the after-removing/adding oil criterion value selected by the information selection section, and
the result output section is configured to output the removing/adding oil amount calculated by the degradation information processing section as a calculation result of the degradation information.

12. An edible oil degradation information processing system, comprising:
an input terminal into which store information, which is information about a store using an edible oil, is input;
a degradation information processing device configured to process degradation information which is information about degradation of the edible oil; and
a notification device configured to notify information output from the degradation information processing device,
the degradation information processing device being configured to:
retain a plurality of pieces of degradation indicator related information which is information about a degradation indicator of the edible oil and associated with the store information;
acquire the store information output from the input terminal;
select the degradation indicator related information corresponding to the store information as acquired, from among the plurality of pieces of degradation indicator related information as stored;
process the degradation information based on the degradation indicator related information as selected; and
output a processing result of the degradation information as processed to the notification device.

13. The edible oil degradation information processing system according to claim 12, further comprising a measurement device for measuring the degradation indicator of the edible oil, wherein
the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively, and
the degradation information processing device is configured to:
further acquire data about a measured value of the degradation indicator measured by the measurement device;
select one of the degradation characteristic models as stored, which corresponds to the store information as acquired, and also select one of the degradation criterion values as stored, which corresponds to the store information as acquired;
predict the degradation information based on the measured value of the degradation indicator as acquired, and the one of the degradation characteristic models and the one of the degradation criterion values as selected; and
output a prediction result of the degradation information as predicted to the notification device.

14. The edible oil degradation information processing system according to claim 12, further comprising a measurement device for measuring the degradation indicator of the edible oil, wherein
the plurality of pieces of degradation indicator related information includes data about degradation criterion values, each of which is indicative of a criterion relating to the degradation indicator, respectively, and
the degradation information processing device is configured to:
further acquire data about a measured value of the degradation indicator measured by the measurement device,
select one of the degradation criterion values as stored, which corresponds to the store information as acquired;
based on the measured value of the degradation indicator as acquired and the one of the degradation criterion values as selected, determine whether the edible oil has reached the one of the degradation criterion values, and
output, as a determination result of the degradation information, a result as determined to the notification device.

15. The edible oil degradation information processing system according to claim 12, wherein
the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil, and
the degradation information processing device is configured to:
acquire data about a measured value of the degradation indicator and remaining oil amount data which is data about a remaining oil amount of the edible oil in the cooking tool immediately before performing the removing/adding oil operation;
select one of the after-removing/adding oil criterion values as stored, which corresponds to the store information as acquired;
calculate a removing/adding oil amount necessary in the removing/adding oil operation, based on the measured value of the degradation indicator and the remaining oil amount data as acquired, and the after-removing/adding oil criterion value as selected; and
output, as a calculation result of the degradation information, the removing/adding oil amount as calculated to the notification device.

16. An edible oil degradation information processing method for processing degradation information which is information about degradation of an edible oil, using an input terminal into which store information, which is information about a store using the edible oil, is input, a degradation information processing device configured to process the degradation information, and a notification device configured to notify information output from the degradation information processing device,
the method comprising:
information acquiring step of, by the degradation information processing device, acquiring the store information output from the input terminal;
information selecting step of, by the degradation information processing device, selecting the degradation indicator related information corresponding to the store information acquired in the information acquiring step, from among the plurality of pieces of degradation indicator related information as stored;
an information processing step of, by the degradation information processing device, processing the degradation information based on the degradation indicator related information selected in the information selecting step; and
a processing result outputting step of, by the degradation information processing device, outputting a processing result of the degradation information processed in the information processing step to the notification device.

17. The edible oil degradation information processing method according to claim 16, further using a measurement device for measuring the degradation indicator of the edible oil, wherein
the plurality of pieces of degradation indicator related information includes data about degradation characteristic models of the edible oil, each of which is indicative of a change in the degradation indicator, and data about degradation criterion values, each of which is indicative of a criterion relating to the degradation indicator, respectively,
in the information acquiring step, the degradation information processing device further acquires data about a measured value of the degradation indicator measured by the measurement device,
in the information selecting step, the degradation information processing device selects one of the degradation characteristic models as stored, which corresponds to the store information acquired in the information acquiring step, and also selects one of the degradation criterion values as stored, which corresponds to the store information acquired in the information acquiring step,
in the information processing step, the degradation information processing device predicts the degradation information based on the measured value of the degradation indicator acquired in the information acquiring step, and the one of the degradation characteristic models and the one of the degradation criterion values selected in the information selecting step, and
in the processing result outputting step, the degradation information processing device outputs a prediction result of the degradation information predicted in the information processing step to the notification device.

18. The edible oil degradation information processing method according to claim 16, further using a measurement device for measuring the degradation indicator of the edible oil, wherein
the plurality of pieces of degradation indicator related information includes data about degradation criterion values, each of which is indicative of a criterion relating to the degradation indicator, respectively,
in the information acquiring step, the degradation information processing device further acquires data about a measured value of the degradation indicator measured by the measurement device,
in the information selecting step, the degradation information processing device selects one of the degradation criterion values as stored, which corresponds to the store information acquired in the information acquiring step,
in the information processing step, based on the measured value of the degradation indicator acquired in the information acquiring step and the one of the degradation criterion values selected in the information selecting step, the degradation information processing device determines whether the edible oil has reached the one of the degradation criterion values, and
in the processing result outputting step, the degradation information processing device outputs, as a determination result of the degradation information, a result as determined in the information processing step to the notification device.

19. The edible oil degradation information processing method according to claim 16, wherein
the plurality of pieces of degradation indicator related information includes data relating to after-removing/adding oil criterion values, each of which serves as a criterion for the degradation indicator and obtained by performing a removing/adding oil operation of removing a part of the edible oil in a cooking tool and adding another edible oil, which is different from the edible oil, in the edible oil,
in the information acquiring step, the degradation information processing device further acquires data about a measured value of the degradation indicator and remaining oil amount data which is data about a remaining oil amount of the edible oil in the cooking tool immediately before performing the removing/adding oil operation,
in the information selecting step, the degradation information processing device selects one of the after-removing/adding oil criterion values as stored, which corresponds to the store information as acquired,
in the information processing step, the degradation information processing device calculates a removing/adding oil amount necessary in the removing/adding oil operation, based on the measured value of the degradation indicator and the remaining oil amount data acquired in the information acquiring step, and the after-removing/adding oil criterion value selected in the information selecting step, and
in the processing result outputting step, the degradation information processing device outputs, as a calculation result of the degradation information, the removing/adding oil amount calculated in the information processing step to the notification device.
